SEQUENCE LISTING (135 sequences)

SEQ ID NO: 1 - DNA, 30 bp, Artificial Sequence
  Description of Artificial Sequence Synthetic oligonucleotide
gtaattgcggcaagaagaattgtttctgtc

SEQ ID NO: 2 - DNA, 30 bp, Artificial Sequence
  Description of Artificial Sequence Synthetic oligonucleotide
gtaattgcggcaggaggaattgtttctgtc

SEQ ID NO: 3 - DNA, 30 bp, Artificial Sequence
  Description of Artificial Sequence Synthetic oligonucleotide
ggcaagagacaccaaagacactaccagcat

SEQ ID NO: 4 - DNA, 27 bp, Artificial Sequence
  Description of Artificial Sequence Synthetic oligonucleotide
tcctgagcaatacctaacgaacaaata

SEQ ID NO: 5 - DNA, 20 bp, Artificial Sequence
  Description of Artificial Sequence Synthetic oligonucleotide
catctccttggtgctcaacc

SEQ ID NO: 6 - DNA, 20 bp, Artificial Sequence
  Description of Artificial Sequence Synthetic oligonucleotide
ctgaagtcgacttggcttgg

SEQ ID NO: 7 - DNA, 21 bp, Artificial Sequence
  Description of Artificial Sequence Synthetic oligonucleotide
ctcagatagtggccagaatgc

SEQ ID NO: 8 - DNA, 20 bp, Artificial Sequence
  Description of Artificial Sequence Synthetic oligonucleotide
ttgtccaggtgactccaagg

SEQ ID NO: 9 - DNA, 25 bp, Artificial Sequence
  Description of Artificial Sequence Synthetic oligonucleotide
cgtccgagcccagaaagatgaggtc

SEQ ID NO: 10 - DNA, 19 bp, Artificial Sequence
  Description of Artificial Sequence Synthetic oligonucleotide
agaggcgcacgtgagacac

SEQ ID NO: 11 - DNA, 19 bp, Artificial Sequence
  Description of Artificial Sequence Synthetic oligonucleotide
tgaagacaatgaagatgtt

SEQ ID NO: 12 - DNA, 25 bp, Artificial Sequence
  Description of Artificial Sequence Synthetic oligonucleotide
tttcttttctatgtgctgttggtga

SEQ ID NO: 13 - protein, 396 aa, Homo sapiens
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDNEEDPMMYPEMELESDKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKVGSKAGSNKSTASSKSGDGKNSVWI

SEQ ID NO: 14 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 15 - protein, 11 aa, Artificial Sequence
  Synthetic connexin peptide
FEVAFLLIQWI

SEQ ID NO: 16 - protein, 11 aa, Artificial Sequence
  Synthetic connexin peptide
LLIQWYIGFSL

SEQ ID NO: 17 - protein, 16 aa, Artificial Sequence
  Synthetic connexin peptide
SLSAVYTCKRDPCPHQ

SEQ ID NO: 18 - protein, 12 aa, Artificial Sequence
  Synthetic connexin peptide
VDCFLSRPTEKT

SEQ ID NO: 19 - protein, 11 aa, Artificial Sequence
  Synthetic connexin peptide
SRPTEKTIFII

SEQ ID NO: 20 - protein, 13 aa, Artificial Sequence
  Synthetic connexin peptide
LGTAVESAWGDEQ

SEQ ID NO: 21 - protein, 12 aa, Artificial Sequence
  Synthetic connexin peptide
QSAFRCNTQQPG

SEQ ID NO: 22 - protein, 12 aa, Artificial Sequence
  Synthetic connexin peptide
QQPGCENVCYDK

SEQ ID NO: 23 - protein, 13 aa, Artificial Sequence
  Synthetic connexin peptide
VCYDKSFPISHVR

SEQ ID NO: 24 - protein, 38 aa, Artificial Sequence
  Synthetic connexin peptide
AAESVWGDEIKSSFICNTLQPGCNSVCYDHFFPISHVR

SEQ ID NO: 25 - protein, 12 aa, Artificial Sequence
  Synthetic connexin peptide
ESVWGDEKSSFI

SEQ ID NO: 26 - protein, 12 aa, Artificial Sequence
  Synthetic connexin peptide
ICNTLQPGCNSV

SEQ ID NO: 27 - protein, 12 aa, Artificial Sequence
  Synthetic connexin peptide
SVCYDHFFPISH

SEQ ID NO: 28 - protein, 25 aa, Artificial Sequence
  Synthetic connexin peptide
RLVKCEAFPCPNTVDCFVSRPTEKT

SEQ ID NO: 29 - protein, 12 aa, Artificial Sequence
  Synthetic connexin peptide
VKCEAFPCPNTV

SEQ ID NO: 30 - protein, 12 aa, Artificial Sequence
  Synthetic connexin peptide
VDCFVSRPTEKT

SEQ ID NO: 31 - protein, 13 aa, Artificial Sequence
  Synthetic connexin peptide
VCYDHFFPISHVR

SEQ ID NO: 32 - protein, 18 aa, Artificial Sequence
  Synthetic connexin peptide
VWGDEKSSFICNTLQPGY

SEQ ID NO: 33 - protein, 15 aa, Artificial Sequence
  Synthetic connexin peptide
DEKSSFICNTLQPGY

SEQ ID NO: 34 - protein, 11 aa, Artificial Sequence
  Synthetic connexin peptide
SRPTEKTVFTV

SEQ ID NO: 35 - protein, 7 aa, Artificial Sequence
  Synthetic connexin peptide
SRPTEKT

SEQ ID NO: 36 - protein, 12 aa, Artificial Sequence
  Synthetic connexin peptide
ICNTLQPGCNSV

SEQ ID NO: 37 - protein, 16 aa, Artificial Sequence
  Synthetic connexin peptide
FLDTLHVCRRSPCPHP

SEQ ID NO: 38 - protein, 18 aa, Artificial Sequence
  Synthetic connexin peptide
KRDPCHQVDCFLSRPTEK

SEQ ID NO: 39 - protein, 35 aa, Artificial Sequence
  Synthetic connexin peptide
KEVWGDEQADFVCNTLQPGCKNVCYDHYFPISHIR

SEQ ID NO: 40 - protein, 35 aa, Artificial Sequence
  Synthetic connexin peptide
QEVWGDEQEDFVCNTLQPGCKNVCYDHFFPVSHIR

SEQ ID NO: 41 - protein, 35 aa, Artificial Sequence
  Synthetic connexin peptide
EEVWDDEQKDFVCNTKQPGCPNVCYDEFFPVSHVR

SEQ ID NO: 42 - protein, 35 aa, Artificial Sequence
  Synthetic connexin peptide
ERVWGDEQKDFDCNTKQPGCTNVCYDNYFPISNIR

SEQ ID NO: 43 - protein, 35 aa, Artificial Sequence
  Synthetic connexin peptide
ERVWSDDHKDFDCNTRQPGCSNVCFDEFFPVSHVR

SEQ ID NO: 44 - protein, 35 aa, Artificial Sequence
  Synthetic connexin peptide
ESVWGDEKSSFICNTLQPGCNSVCYDQFFPISHVR

SEQ ID NO: 45 - protein, 35 aa, Artificial Sequence
  Synthetic connexin peptide
ESVWGDEQSDFECNTAQPGCTNVCYDQAFPISHIR

SEQ ID NO: 46 - protein, 35 aa, Artificial Sequence; identical to SEQ ID NO: 45
  Synthetic connexin peptide

SEQ ID NO: 47 - protein, 35 aa, Artificial Sequence
  Synthetic connexin peptide
RPVYQDEQERFVCNTLQPGCANVCYDVFSPVSHLR

SEQ ID NO: 48 - protein, 35 aa, Artificial Sequence
  Synthetic connexin peptide
ESAWGDEQSAFRCNTQQPGCENVCYDKSFPISHVR

SEQ ID NO: 49 - protein, 35 aa, Artificial Sequence
  Synthetic connexin peptide
EDVWGDEQSDFTCNTQQPGCBNVCYBRAFPISHIR

SEQ ID NO: 50 - protein, 35 aa, Artificial Sequence
  Synthetic connexin peptide
EAIYSDEQAKFTCNTRQPGCDNVCYDAFAPLSHVR

SEQ ID NO: 51 - protein, 35 aa, Artificial Sequence
  Synthetic connexin peptide
ESSWGDEQADFRCDTIQPGCQNVCTDQAFPISHIR

SEQ ID NO: 52 - protein, 36 aa, Artificial Sequence
  Synthetic connexin peptide
GESIYYDEQSKFVCNTEQPGCENVCYDAFAPLSHVR

SEQ ID NO: 53 - protein, 39 aa, Artificial Sequence
  Synthetic connexin peptide
MYVFYVMYDGFSMQRLVKCNAWPCPNTVDCFVSRPTEKT

SEQ ID NO: 54 - protein, 39 aa, Artificial Sequence
  Synthetic connexin peptide
MYVFYFLYNGYHLPWVLKCGIDPCPNLVDCFISRPTEKT

SEQ ID NO: 55 - protein, 39 aa, Artificial Sequence
  Synthetic connexin peptide
LYIFHRLYKDYDMPRVVACSVEPCPHTVDCYISRPTEKK

SEQ ID NO: 56 - protein, 40 aa, Artificial Sequence
  Synthetic connexin peptide
LYLLHTLWHGFNMPRLVQCANVAPCPNIVDCYIARPTEKK

SEQ ID NO: 57 - protein, 39 aa, Artificial Sequence
  Synthetic connexin peptide
LYVFHSFYPKYILPPVVKCHADPCPNIVDCFISKPSEKN

SEQ ID NO: 58 - protein, 39 aa, Artificial Sequence
  Synthetic connexin peptide
MYVFYLLYPGYAMVRLVKCDVYPCPNTVDCFVSRPTEKT

SEQ ID NO: 59 - protein, 32 aa, Artificial Sequence
  Synthetic connexin peptide
LYGWTMEPVFVCQRAPCPYLVDCFVSRPTEKT

SEQ ID NO: 60 - protein, 32 aa, Artificial Sequence; identical to SEQ ID NO: 59
  Synthetic connexin peptide

SEQ ID NO: 61 - protein, 38 aa, Artificial Sequence
  Synthetic connexin peptide
GALHYFLFGFLAPKKFPCTRPPCTGVVDCYVSRPTSKS

SEQ ID NO: 62 - protein, 38 aa, Artificial Sequence
  Synthetic connexin peptide
LLIQWYIYGFSLSAVYTCKRDPCPHQVDCFLSRPTEKT

SEQ ID NO: 63 - protein, 38 aa, Artificial Sequence
  Synthetic connexin peptide
IAGQYFLYGFELKPLYRCDRWPCPNTVDCFISRPTEKT

SEQ ID NO: 64 - protein, 38 aa, Artificial Sequence
  Synthetic connexin peptide
LVGQYLLYGFEVRPFFPCSRQPCPHVVDCFVSRPTEKT

SEQ ID NO: 65 - protein, 38 aa, Artificial Sequence
  Synthetic connexin peptide
IVGQYFIYGIFLTTLHVCRRSPCPHPVNCYVSRPTEKN

SEQ ID NO: 66 - protein, 38 aa, Artificial Sequence
  Synthetic connexin peptide
LIGQYFLYGFQVHPFYVCSRLPCHPKIDCFISRPTEKT

SEQ ID NO: 67 - protein, 42 aa, Artificial Sequence
  Synthetic connexin peptide
LLIQWYIYGFSLSAVYTCKRDPCPHQVDCFLSRPTEKTIFII

SEQ ID NO: 68 - protein, 43 aa, Artificial Sequence
  Synthetic connexin peptide
MYVFYVMYDGFSMQRLVKCNAWPCPNTVDCFVSRPTEKTVFTV

SEQ ID NO: 69 - protein, 42 aa, Artificial Sequence
  Synthetic connexin peptide
YVFYFLYNGYHLPWVLKCGIDPCPNLVDCFISRPTEKTVFTI

SEQ ID NO: 70 - protein, 43 aa, Artificial Sequence
  Synthetic connexin peptide
LYIFHRLYKDYDMPRVVACSVEPCPHTVDCYISRPTEKKVFTY

SEQ ID NO: 71 - protein, 42 aa, Artificial Sequence
  Synthetic connexin peptide
LYLLHTLWHGFNMPRLVQCANVAPCPNIVDCYIARPTEKKTY

SEQ ID NO: 72 - protein, 43 aa, Artificial Sequence
  Synthetic connexin peptide
LYVFHSFYPKYILPPVVKCHADPCPNIVDCFISKPSEKNIFTL

SEQ ID NO: 73 - protein, 43 aa, Artificial Sequence
  Synthetic connexin peptide
MYVFYLLYPGYAMVRLVKCDVYPCPNTVDCFVSRPTEKTVFTV

SEQ ID NO: 74 - protein, 36 aa, Artificial Sequence
  Synthetic connexin peptide
LYGWTMEPVFVCQRAPCPYLVDCFVSRPTEKTIFII

SEQ ID NO: 75 - protein, 36 aa, Artificial Sequence; identical to SEQ ID NO: 74
  Synthetic connexin peptide

SEQ ID NO: 76 - protein, 42 aa, Artificial Sequence
  Synthetic connexin peptide
GALHYFLFGFLAPKKFPCTRPPCTGVVDCYVSRPTEKSLLML

SEQ ID NO: 77 - protein, 42 aa, Artificial Sequence
  Synthetic connexin peptide
IAGQYFLYGFELKPLYRCDRWPCPNTVDCFISRPTEKTIFII

SEQ ID NO: 78 - protein, 42 aa, Artificial Sequence
  Synthetic connexin peptide
LVGQYLLYGFEVRPFFPCSRQPCPHVVDCFVSRPTEKTVFLL

SEQ ID NO: 79 - protein, 41 aa, Artificial Sequence
  Synthetic connexin peptide
IVGQYFIYGIFLTTLHVCRRSPCPHPVNCYSRPTEKNVFIV

SEQ ID NO: 80 - protein, 42 aa, Artificial Sequence
  Synthetic connexin peptide
LIGQYFLYGFQVHPFYVCSRLPCHPKIDCFISRPTEKTIFLL

SEQ ID NO: 81 - protein, 45 aa, Artificial Sequence
  Synthetic connexin peptide
LGTAAESSWGDEQADFRCDTIQPGCQNVCTDQAFPISHIRFWVLQ

SEQ ID NO: 82 - protein, 14 aa, Artificial Sequence
  Synthetic connexin peptide
LGTAAESSWGDEQA

SEQ ID NO: 83 - protein, 13 aa, Artificial Sequence
  Synthetic connexin peptide
DEQADFRCDTIQP

SEQ ID NO: 84 - protein, 13 aa, Artificial Sequence
  Synthetic connexin peptide
TIQPGCQNVCTDQ

SEQ ID NO: 85 - protein, 13 aa, Artificial Sequence
  Synthetic connexin peptide
VCTDQAFPISHIR

SEQ ID NO: 86 - protein, 13 aa, Artificial Sequence
  Synthetic connexin peptide
AFPISHIRFWVLQ

SEQ ID NO: 87 - protein, 47 aa, Artificial Sequence
  Synthetic connexin peptide
MEVGFIVGQYFIYGIFLTTLHVCRRSPCPHPVNCYVSRPTEKNVFIV

SEQ ID NO: 88 - protein, 11 aa, Artificial Sequence
  Synthetic connexin peptide
MEVGFIVGQYF

SEQ ID NO: 89 - protein, 12 aa, Artificial Sequence
  Synthetic connexin peptide
IVGQYFIYGIFL

SEQ ID NO: 90 - protein, 14 aa, Artificial Sequence
  Synthetic connexin peptide
GIFLTTLHVCRRSP

SEQ ID NO: 91 - protein, 12 aa, Artificial Sequence
  Synthetic connexin peptide
RRSPCPHPVNCY

SEQ ID NO: 92 - protein, 12 aa, Artificial Sequence
  Synthetic connexin peptide
VNCYVSRPTEKN

SEQ ID NO: 93 - protein, 11 aa, Artificial Sequence
  Synthetic connexin peptide
SRPTEKNVFIV

SEQ ID NO: 94 - protein, 46 aa, Artificial Sequence
  Synthetic connexin peptide
LTAVGGESIYYDEQSKFVCNTEQPGCENVCYDAFAPLSHVRFWVFQ

SEQ ID NO: 95 - protein, 15 aa, Artificial Sequence
  Synthetic connexin peptide
LTAVGGESIYYDEQS

SEQ ID NO: 96 - protein, 13 aa, Artificial Sequence
  Synthetic connexin peptide
DEQSKFVCNTEQP

SEQ ID NO: 97 - protein, 13 aa, Artificial Sequence
  Synthetic connexin peptide
TEQPGCENVCYDA

SEQ ID NO: 98 - protein, 13 aa, Artificial Sequence
  Synthetic connexin peptide
VCYDAFAPLSHVR

SEQ ID NO: 99 - protein, 12 aa, Artificial Sequence
  Synthetic connexin peptide
APLSHVRFWVFQ

SEQ ID NO: 100 - protein, 47 aa, Artificial Sequence
  Synthetic connexin peptide
FEVGFLIGQYFLYGFQVHPFYVCSRLPCHPKIDCFISRPTEKTIFLL

SEQ ID NO: 101 - protein, 11 aa, Artificial Sequence
  Synthetic connexin peptide
FEVGFLIGQYF

SEQ ID NO: 102 - protein, 12 aa, Artificial Sequence
  Synthetic connexin peptide
LIGQYFLYGFQV

SEQ ID NO: 103 - protein, 14 aa, Artificial Sequence
  Synthetic connexin peptide
GFQVHPFYVCSRLP

SEQ ID NO: 104 - protein, 12 aa, Artificial Sequence
  Synthetic connexin peptide
SRLPCHPKIDCF

SEQ ID NO: 105 - protein, 12 aa, Artificial Sequence
  Synthetic connexin peptide
IDCFISRPTEKT

SEQ ID NO: 106 - protein, 11 aa, Artificial Sequence
  Synthetic connexin peptide
SRPTEKTIFLL

SEQ ID NO: 107 - protein, 11 aa, Artificial Sequence
  Synthetic connexin peptide
SRGGEKNVFIV

SEQ ID NO: 108 - protein, 10 aa, Artificial Sequence
  Synthetic connexin peptide
YVCSRLPCHP

SEQ ID NO: 109 - protein, 11 aa, Artificial Sequence
  Synthetic connexin peptide
QVHPFYVCSRL

SEQ ID NO: 110 - protein, 13 aa, Artificial Sequence
  Synthetic connexin peptide
FEVGFLIGQYFLY

SEQ ID NO: 111 - protein, 12 aa, Artificial Sequence
  Synthetic connexin peptide
GQYFLYGFQVHP

SEQ ID NO: 112 - protein, 12 aa, Artificial Sequence
  Synthetic connexin peptide
GFQVHPFYVCSR

SEQ ID NO: 113 - protein, 12 aa, Artificial Sequence
  Synthetic connexin peptide
AVGGESIYYDEQ

SEQ ID NO: 114 - protein, 12 aa, Artificial Sequence
  Synthetic connexin peptide
YDEQSKFVCNTE

SEQ ID NO: 115 - protein, 12 aa, Artificial Sequence
  Synthetic connexin peptide
NTEQPGCENVCY

SEQ ID NO: 116 - protein, 12 aa, Artificial Sequence
  Synthetic connexin peptide
CYDAFAPLSHVR

SEQ ID NO: 117 - protein, 12 aa, Artificial Sequence
  Synthetic connexin peptide
FAPLSHVRFWVF

SEQ ID NO: 118 - protein, 5 aa, Artificial Sequence
  Synthetic connexin peptide
LIGQY

SEQ ID NO: 119 - protein, 5 aa, Artificial Sequence
  Synthetic connexin peptide
QVHPF

SEQ ID NO: 120 - protein, 5 aa, Artificial Sequence
  Synthetic connexin peptide
YVCSR

SEQ ID NO: 121 - protein, 5 aa, Artificial Sequence
  Synthetic connexin peptide
SRLPC

SEQ ID NO: 122 - protein, 5 aa, Artificial Sequence
  Synthetic connexin peptide
LPCHP

SEQ ID NO: 123 - protein, 5 aa, Artificial Sequence
  Synthetic connexin peptide
GESIY

SEQ ID NO: 124 - protein, 6 aa, Artificial Sequence
  Synthetic connexin peptide
YDEQSK

SEQ ID NO: 125 - protein, 6 aa, Artificial Sequence
  Synthetic connexin peptide
SKFVCN

SEQ ID NO: 126 - protein, 8 aa, Artificial Sequence
  Synthetic connexin peptide
TEQPGCEN

SEQ ID NO: 127 - protein, 8 aa, Artificial Sequence
  Synthetic connexin peptide
VCYDAFAP

SEQ ID NO: 128 - protein, 10 aa, Artificial Sequence
  Synthetic connexin peptide
LSHVRFWVFQ

SEQ ID NO: 129 - protein, 14 aa, Artificial Sequence
  Synthetic connexin peptide
LIQYFLYGFQVHPF

SEQ ID NO: 130 - protein, 13 aa, Artificial Sequence
  Synthetic connexin peptide
VHPFYCSRLPCHP

SEQ ID NO: 131 - protein, 22 aa, Artificial Sequence
  Synthetic connexin peptide
VGGESIYYDEQSKFVCNTEQPG

SEQ ID NO: 132 - protein, 22 aa, Artificial Sequence
  Synthetic connexin peptide
TEQPGCENVCYDAFAPLSHVRF

SEQ ID NO: 133 - protein, 14 aa, Artificial Sequence
  Synthetic connexin peptide
AFAPLSHVRFWVFQ

SEQ ID NO: 134 - DNA, 1314 bp, Homo sapiens
ggcttttagcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 135 - DNA, 1149 bp, Homo sapiens
atgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

(19) United States
(12) Patent Application Publication
(10) Pub. No.: US 2011/0223204 A1
(43) Pub. Date: Sep. 15, 2011

(54) TREATMENT OF PAIN WITH GAP JUNCTION MODULATION COMPOUNDS

(76) Inventors: Bradford J Duft, Rancho Santa Fe, CA (US); Colin Richard Green, Auckland (NZ)

(21) Appl. No.: 12/996,359

(22) PCT Filed: Jun. 4, 2009
(86) PCT No.: PCT/US2009/003408

§ 371 Date: Apr. 1, 2011

Related U.S. Application Data
(60) Provisional application No. 61/131,127, filed on Jun. 4, 2008.

(51) Int. Cl. A61K 31/7088; A61K 38/02; A61K 9/00; A61P 29/00

(52) U.S. Cl. 424/400; 514/44 R; 514/44 A; 514/1.2

(57) ABSTRACT

The present invention relates to delivery, including transdermal delivery, of a therapeutically effective amount of a compound useful for modulating gap junction formation and function, including an oligonucleotide for reducing gap junction formation and function, and methods and compositions for treating a subject suffering from pain associated with a disease, disorder or condition and associated with pain, including but not limited to muscle pain, ligament pain, tendon pain, joint pain and post-operative pain.

### FIELD

The field involves the delivery of compounds useful for pain relief by modulation of gap junctions, including oligonucleotide gap junction modulators, through the skin.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

Today, pain has become the universal disorder, a serious and costly public health issue, and a challenge for family, friends, and health care providers who must give support to the individual suffering from the physical as well as the emotional consequences of pain. The International Association for the Study of Pain defines it as: “An unpleasant sensory and emotional experience associated with actual or potential tissue damage or described in terms of such damage.” In general, there are two basic types of pain, acute and chronic. Acute pain, for the most part, results from disease, inflammation, or injury to tissues. This type of pain generally comes on suddenly, for example, after trauma or surgery, and may be accompanied by anxiety or emotional distress. In some instances, it can become chronic. Chronic pain is widely believed to represent disease itself. Chronic pain persists over a longer period of time than acute pain and is resistant to most medical treatments. It can, and often does, cause severe problems for patients.

Arthritis is considered to be one of the most pervasive diseases in the United States and a leading cause of disability. According to the Centers for Disease Control and Prevention, it is estimated that 1 of every 3 Americans is affected by the more than 100 types of arthritis. Pain, particularly of the joints throughout the body, characterizes arthritis. Psoriasis, primarily a skin disorder, can progress to psoriatic arthritis if left untreated. Rheumatoid arthritis, osteoarthritis, and ankylosing spondylitis are all examples of degenerative arthritic diseases.

In addition to, for example, arthritic causes, normal function of a joint and its movement, and other portions of the body, can be severely impaired as a result of trauma or following orthopedic and other surgical procedures. This may result in tenderness, aching, pain, and lengthy recovery times, as well as the loss of joint mobility or reduced range of motion, tonicity, or elasticity of the joint/articular structures, such as for example, muscle, tendon, capsule, bone, or ligament. Reduced joint mobility may also involve permanently altered or shortened joint or tissue architecture. Altered or abnormal joint mobility or joint architecture may also be associated with or caused by a variety of injuries and conditions such as, for example, metabolic disorders, ischemia, injury to joint, capsule, bone, cartilage, tendon, ligament or muscle, fractures, subluxation, dislocation, crush injuries, prolonged immobilization (e.g., immobilization of a joint in a cast or splint), and paralysis. To date, common surgical interventions to alleviate altered or abnormal joint mobility or joint architecture have met with limited success as corrective surgical procedure is also a form of controlled injury or trauma and the procedure can cause further pain.

Gap junctions are cell membrane structures that facilitate direct cell-cell communication. A gap junction channel is formed of two connexons (hemichannels), each composed of six connexin subunits. Each hexameric connexon docks with a connexon in the opposing membrane to form a single gap junction. Gap junction channels are reported to be found throughout the body. Tissue such as the corneal epithelium, for example, has six to eight cell layers, yet expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues such as cornea have been shown to alter connexin protein expression pattern following injury or transplantation (Qui, C. et al., (2003) Current Biology, 13:1967-1703; Brander et al., (2004), J. Invest Dermatol. 122:1310-20).

It has been reported that abnormal connexin function may be linked to certain disease states (e.g. heart diseases) (A. C. de Carvalho, et al., J Cardiovasc Electrophysiol 5:686 (1994)). In certain connexin proteins, alterations in the turnover and trafficking properties may be induced by the addition exogenous agents which may affect the level of gap junctional intercellular communication (Darrow, B. J., et al., Circ Res 76:381 (1995); Lin R, et al., J Cell Biol 154(4):815 (2001). Antisense technology has been reported for the modulation of the expression for genes implicated in viral, fungal and metabolic diseases. See, e.g., U.S. Pat. No. 5,166,195, (oligonucleotide inhibitors of HIV), U.S. Pat. No. 5,004,810 (oligomers for hybridizing to herpes simplex virus Vmw65 mRNA and inhibiting replication). See also U.S. Pat. No. 7,098,190 to Becker and Green (formulations comprising antisense nucleotides to connexins). Peptide inhibitors (including peptidomimetics) of gap junctions and hemichannels have been reported. See for example Berthoud, V. M. et al., Am J. Physiol. Lung Cell Mot Physiol. 279: L619-L622 (2000); Evans, W. H. and Boitano, S. Biochem. Soc. Trans. 29: 606-612, and De Vriese A. S., et al. Kidney Int. 61: 177-185 (2001). See also Green and Becker, WO2006/134494 (“Anti-connexin compounds and methods of use”).

Despite advances in the understanding of the principles of the mechanisms underlying the processes related to pain and causation of pain relief (including pain associated with arthritic conditions and surgical and orthopedic procedures), there remains a significant unmet need for suitable therapeutic options for improving outcomes and recoveries.

The skin provides a protective barrier against foreign materials and infection. In mammals this is accomplished by forming a highly insoluble protein and lipid structure on the surface of corneocytes, called the cornified envelope (CE). (Downing et al., Dermatology in General Medicine, Fitzpatrick, et al., eds., pp. 210-221 (1993), Ponec, M., The Keratinocyte Handbook, Leigh, et al., eds., pp. 351-363 (1994)). The CE is composed of polar lipids, such as ceramides, sterols, and fatty acids, and a complicated network of cross-linked proteins; however, the cytoplasm of stratum corneum cells remains polar and aqueous. The CE is extremely thin (10 microns) but provides a substantial barrier. Nevertheless, the skin has been considered as a route for the administration of drugs. Most transdermal delivery systems achieve epidermal penetration by using a skin penetration enhancing vehicle. Such compounds or mixtures of compounds are known in the art by various terms including, for example, as “penetration enhancers” or “skin enhancers”. Other methods for transdermal delivery of therapeutic compounds include devices, such as ionophoretic, electroporation, and micropenetration devices.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

One aspect of the invention provides novel treatments of conditions associated with pain by application of gap junction modulation agents to the skin. Gap junction modulation agents include anti-connexin compounds, gap junction modifying compounds, connexin binding compounds, and hemichannel modulation compounds. Another aspect of the present invention is to relieve pain by application of anti-connexin compounds to or into the skin. In one embodiment skin pain is reduced. In another embodiment, pain caused by or due to trauma is reduced. In one embodiment, pain is reduced in a supporting body structure of a subject, including (alone, together, or in any combination) joints, muscles, tendons, ligaments, cartilage and skin, by topically administering to a subject in need thereof a pharmaceutical composition comprising a therapeutically effective amount of a gap junction modulation agent, such as an anti-connexin 43 compound, in a pharmaceutically acceptable transdermal delivery form or device, whereby pain is reduced. In another embodiment, pain in the musculoskeletal system of a subject is reduced. In another embodiment, pain in a supporting body structure of a subject and/or in the musculoskeletal system of a subject is reduced by injecting or instilling a pharmaceutical composition comprising a therapeutically effective amount of a gap junction modulation agent, such as an anti-connexin 43 compound, including, for example, injection or instillation of a depot formulation, or a slow, sustained or delayed release formulation thereof.

Aspects of the invention concern transdermal formulations and devices for delivery of gap junction modulation agents.

According to a non-limiting preferred aspect, the connexin modulated is a connexin 43 and the connexin 43 gap junction or hemichannel modulated is a connexin 43 gap junction or hemichannel.

In one embodiment, the gap junction modulation agent is an anti-connexin polynucleotide, preferably an anti-connexin oligonucleotide. In another embodiment the anti-connexin oligonucleotide is an antisense oligonucleotide. In other embodiments the anti-connexin oligonucleotide is an RNAi or an siRNA compound. According to an alternate embodiment the anti-connexin oligonucleotide is a ribozyme compound. In certain non-limiting preferred embodiments, the anti-connexin oligonucleotide is an anti-connexin 43 oligonucleotide.

In other embodiments, the gap junction modulation agent is a peptide or polypeptide, an antibody or binding fragment thereof, a peptidomimetic, a peptide analog or a connexin carboxy-terminal polypeptide. Non-limiting preferred peptides and peptidomimetics include anti-connexin 43 peptides or peptidomimetics, including anti-connexin 43 hemichannel blocking peptides or anti-connexin 43 hemichannel blocking peptidomimetics. Non-limiting preferred connexin carboxy-terminal polypeptides include connexin 43 carboxy-terminal polypeptides. Other non-limiting preferred gap junction modulation agents include anti-connexin compounds, connexin binding compounds and hemichannel modulation compounds such as anti-connexin 43 compounds, anti-connexin 43 binding compounds and connexin 43 hemichannel modulation compounds.

In other embodiments, the gap junction modulation agent is a gap junction modifying compound (including, for example, connexin protein phosphorylation agents that restrict or close gap junctions), a connexin binding compound (including, for example, connexin carboxy-terminal polypeptides that block or inhibit ZO-1 protein interaction), a hemichannel modulation compound (including, for example, mimetic peptides that can bind to and restrict connexin hemichannel opening), or an anti-ZO-1 protein oligonucleotide. Non-limiting preferred gap junction modifying compounds include connexin 43 gap junction modifying compounds. Non-limiting preferred connexin binding compounds include connexin 43 binding compounds. Non-limiting preferred hemichannel modulation compound include connexin 43 hemichannel modulation compounds. Non-limiting preferred anti-ZO-1 protein oligonucleotides include those useful for modulating connexin 43 activity or otherwise binding to connexin 43.

Various uses of gap junction modulation agents include uses for treatment, or in the manufacture or preparation of formulations, compositions, articles of manufacture or kits.

For example, embodiments of the transdermal delivery system for delivery of gap junction modulation agents include formulations that deliver a therapeutically effective amount of a gap junction modulation agent by application to the skin. Methods of making the transdermal delivery systems described herein and methods of using said formulation (e.g., the treatment and prevention of pain), are further embodiments.

In still other embodiments, a transdermal formulation for use in the invention comprises a lipid composition to enhance transdermal penetration. Such lipid compositions include a vegetable, nut, animal, or synthetic oil or fatty acid, fatty alcohol, or fatty amine. Non-limiting preferred oils include a macadamia nut oil, meadowfoam oil (limnanthes alba), castor oil, jojoba oil, corn oil, sunflower oil, sesame oil or an emu oil. One especially preferred oil is an emu oil.

In certain non-limiting preferred embodiments, the transdermal delivery system comprises an ethoxylated oil or fatty acid, fatty alcohol, or fatty amine therein having about 10 to 19 ethoxylations per molecule. Ethoxylated lipids suitable as a penetration enhancer include oils such as an ethoxylated vegetable, nut, synthetic or animal oil, suitably ethoxylated emu oil or ethoxylated macadamia nut oil. According to a non-limiting preferred aspect, suitable ethoxylated lipids that can be used in the formulations described herein can be a vegetable, nut, animal, or synthetic oil or fatty acid, fatty alcohol, or fatty amine therein having at least 10, 11, 12, 13, 14, 15, 16, 17, 18, 19, or more ethoxylations per molecule. Non-limiting preferred ethoxylated oils include macadamia nut oil, meadowfoam oil (limnanthes alba) castor oil, jojoba oil, corn oil, sunflower oil, sesame oil or emu oil. Optionally, other conventional agents used in pharmaceutical formulations such as an alcohol and/or water and/or an aqueous adjuvant can be mixed with the penetration enhancer to improve the solubility and/or transport of a particular gap junction modulation agent.

In some embodiments, the transdermal delivery systems described herein are suitable for transdermal administration of gap junction modulation agents that are molecules with a molecular weight equal to or less than about 9,000 to about 10,000 daltons. In some embodiments, however, the gap junction modulation agent is a molecule with a molecular weight equal to or greater than about 9,000 to about 10,000 daltons.

In still other embodiments, the transdermal delivery system comprises a microneedle, microprojection array or other micropenetration device in combination with one or more gap junction modulation agents. Non-limiting preferred anti-connexin compounds include anti-connexin 43 compounds. Non-limiting preferred gap junction modifying agents include those that modulate connexin 43 gap junctions. Non-limiting preferred connexin binding agents include connexin 43 binding compounds. Non-limiting preferred hemichannel modulation agents include those that modulate connexin 43 hemichannels. Other transdermal delivery system include electroporation, iontophoresis, sonophoresis, and ultrasound devices comprising a gap junction modulation agent, preferably a connexin 43 gap junction modulation agent.

Several methods of using the transdermal delivery formulations and devices are also embodiments. For example, one approach involves a method of reducing pain by transdermal delivery of a formulation that comprises a gap junction modulation agent in the treatment of a subject in need of a reduction of pain. Monitoring the reduction in pain may also be desired as part of a treatment or rehabilitation program.

The invention also includes transdermal delivery compositions, useful for pain relief or prevention in the treatment of a subject, including in the treatment of a subject for an arthritic condition or during or following (and/or before, as a pretreatment) an invasive medical procedure or surgery, including an orthopedic procedure or surgery, or a subject predisposed to or otherwise at risk for pain, comprising one or more gap junction modulation agents. The invention also includes compositions for transdermal delivery of a gap junction modulation agent, useful for the treatment of such subjects in need of treatment. Agents and formulations described herein are administered to a site of pain (acute or chronic, for example, or for the prevention of pain) and/or proximally thereto (including, for example, areas of reflected or secondary pain). Thus, for example, agents and formulations are administered to a site of pain and/or to locations proximal thereto in a supporting body structure of a subject, including joints, muscles, tendons, ligaments, cartilage and skin (including any one or more of these, together, or in any combination), and/or in the musculoskeletal system, by topical or other administration as provided herein (including, for example, by injection or instillation), whereby pain is reduced.

Treatment of a subject to provide pain relief using a transdermal delivery composition, method and/or a transdermal delivery device, which may comprise one or more gap junction modulation agents, may involve combined, simultaneous, separate, sequential or sustained administration of such composition. Multiple applications are also provided for relief or prevention of pain.

According to some aspects, the invention generally relates to the use (including for use in treatment or in the manufacture or preparation of compositions, formulations, articles of manufacture, and kits) of one or more gap junction modulation agents, to provide pain relief or pain prevention for the treatment of a subject suffering therefrom, predisposed to, or at risk thereof. Uses for pre- and/or post-surgical patients not only provide pain relief, but also improved recovery and accelerated recovery times.

In one aspect, the invention includes a transdermal delivery composition comprising a pharmaceutically acceptable gap junction modulation agent, for pain relief or prevention in the treatment of a subject, including in the treatment of a subject for an arthritic condition or during or following (and/or before, as a pretreatment) an invasive medical procedure or surgery, including an orthopedic procedure or surgery, for example.

In other embodiments, two or more gap junction modulation agents, including sub-therapeutically effective amounts of two or more gap junction modulation agents, may be used for administration by transdermal delivery separately or jointly to provide a combined action that is therapeutically effective. Thus, compositions for transdermal delivery useful for the prophylactic or affirmative treatment of a subject for pain are also provided in the form of a single gap junction modulation agent or a combined preparation, for example, as an admixture of therapeutically effective amounts of two or more gap junction modulation agents, for example one or more anti-connexin polynucleotides and one or more anti-connexin peptides, peptidomimetics, or gap junction modifying agents. In other embodiments, sub-therapeutically effective amounts of two or more gap junction modulation agents are administered by transdermal delivery in combination to provide a desired therapeutically effect.

In one embodiment, a composition comprising one or more anti-connexin polynucleotides is administered by transdermal delivery at or about the same time as transdermal delivery by one or more anti-connexin peptides, peptidomimetics, or gap junction modifying agents, or other gap junction modulation agents, for example. In one embodiment, a transdermal delivery composition comprising one or more anti-connexin polynucleotides is administered within at least about thirty minutes of one or more anti-connexin peptides, peptidomimetics, or gap junction modifying agents or other gap junction modulation agents. In one embodiment, a transdermal delivery composition comprising one or more anti-connexin polynucleotides is administered within at least about one hour of a transdermal delivery composition of one or more anti-connexin peptides, peptidomimetics, or gap junction modifying agents or other gap junction modulation agents. In one embodiment, a transdermal delivery composition comprising one or more anti-connexin polynucleotides is administered within at least about 2 to 12 hours of a transdermal delivery composition comprising one or more anti-connexin peptides, peptidomimetics, or gap junction modifying agents or other gap junction modulation agents. In one embodiment, a transdermal delivery composition comprising one or more anti-connexin polynucleotides is administered within at least about 24 to 48 hours of transdermal delivery of one or more anti-connexin peptides, peptidomimetics, or gap junction modifying agents or other gap junction modulation agents. In another embodiment the anti-connexin polynucleotide and anti-connexin peptide or peptidomimetic or other gap junction modulation agent are administered by transdermal delivery within about 1 to 8 hours of each other, within about one day of each other, or within about one week of each other. Other embodiments include administration by transdermal delivery of one or more anti-connexin polynucleotides and/or one or more anti-connexin peptides, peptidomimetics, or gap junction modifying agents, and one or more gap junction closing compounds, one or more hemichannel closing compounds, and/or one or more connexin carboxy-terminal polypeptides. The gap junction modulation agents may be administered in any order.

The invention includes methods for the use of a therapeutically effective amount of one or more gap junction modulation agents as described herein, in the manufacture of a dosage form (including a device comprising a dosage form) suitable for transdermal delivery and useful for treating a subject to provide pain relief. Such dosage forms and devices include those for the treatment of a subject as disclosed herein.

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In certain other aspect, the invention provides: a package comprising therapeutically effective amounts of one or more gap junction modulation agents in a formulation suitable for transdermal delivery together with instructions for use alone or together with one or more other gap junction modulation agents (or a combination thereof). In other embodiments, the package contains sub-therapeutically effective amounts of one or more gap junction modulation agents that, when used together or in combination provide are therapeutically effective.

In one aspect, the present invention is directed to a method for reducing pain in a supporting body structure of a subject, comprising topically administering to said subject in need thereof a pharmaceutical composition comprising a therapeutically effective amount of a connexin 43 gap junction modulation agent in a pharmaceutically acceptable transdermal delivery form, whereby pain is reduced. According to one embodiment, the supporting body structure is a joint. According to another embodiment, the supporting body structure is selected from the group consisting of muscles, bones, tendons, ligaments and cartilage. These methods are suitable for treating a subject suffering from arthritis. Conditions which may be treated include osteoarthritis, rheumatoid arthritis, cervical arthritis; and ankylosing spondylitis.

In a further embodiment this method is suitable for treating a subject suffering from acute pain. Suitable pain conditions for treatment by this method include, back pain, knee pain, hip pain, shoulder pain, hand pain or finger pain. In an alternate embodiment, the subject is suffering from chronic pain, and may include back pain, knee pain, hip pain, shoulder pain, hand pain or finger pain. In another embodiment, the subject is suffering from postoperative pain.

Suitable transdermal dosage forms include a topical gel, lotion, ointment, or spray.

In one aspect, said transdermal delivery form comprises a transdermal penetration agent comprising an oil. Suitably, the oil is an ethoxylated oil having between 10 and 19 ethoxylations/molecule. Suitably, said ethoxylated oil is an ethoxylated emu oil. According to an alternate preferred aspect, the oil comprises an oil selected from the group consisting of macadamia nut oil, meadowfoam oil, castor oil, jojoba oil, corn oil, sun flower oil, sesame oil and emu oil.

In an alternate embodiment said connexin 43 gap junction modulation agent is 10,000 daltons or greater. Alternatively, said connexin 43 gap junction modulation agent is less than 10,000 daltons.

In an embodiment said connexin 43 gap junction modulation agent is an oligonucleotide. Suitable oligonucleotides include those selected from the group consisting of an antisense oligonucleotide, a ribozyme, a RNAi oligonucleotide and a siRNA oligonucleotide.

In one aspect, the present invention is directed to methods wherein said connexin 43 gap junction modulation agent is a connexin 43 antisense oligonucleotide. A suitable antisense oligonucleotide include GTA ATT GCG GCA AGA AGA ATT GTT TCT GTC (SEQ ID NO:1); GTA ATT GCG GCA GGA GGA ATT GTT TCT GTC (SEQ ID NO:2); and GGC AAG AGA CAC CAA AGA CAC TAC CAG CAT (SEQ ID NO:3).

Alternatively, suitable antisense oligonucleotides have from about 15 to about 35 nucleotides and are sufficiently complementary to connexin 43 mRNA to form a duplex having a melting point greater than 20° C. under physiological conditions. Other suitable antisense oligonucleotides have from about 15 to about 35 nucleotides and have at least about 70 percent homology to an antisense sequence of connexin 43 mRNA.

Other suitable connexin 43 gap junction modulation agents include a RNAi or siRNA polynucleotides.

Alternatively, said connexin 43 gap junction modulation agent is a peptide or peptidomimetic. In one aspect, said peptide or peptidomimetic binds to a connexin 43 hemichannel.

In another aspect, said peptide or peptidomimetic binds to a connexin 43 ZO-1 protein binding site.

Additional suitable connexin 43 gap junction modulation agents include a connexin 43 phosphorylation agent.

In a further embodiment, provided are methods according to the present invention further comprising a second pharmaceutical compound, wherein said second pharmaceutical compound is a non-steroidal anti inflammatory drug, e.g., diclofenac.

The compositions of the present invention are conveniently administered to skin proximal to a site of tissue or joint pain in the subject.

Also provided is a pharmaceutical composition for reducing pain in a subject, comprising a pain-reducing amount of, for example, an anti-connexin 43 compound and a pharmaceutically acceptable vehicle comprising a transdermal delivery agent. Additionally, provided is a pharmaceutical composition for reducing pain in a supporting body structure of a subject, comprising a formulation having a pain-reducing amount of, for example, an anti-connexin 43 compound in a transdermal dosage form. Optionally, the composition comprises a transdermal penetration enhancer. In other pharmaceutical compositions, said anti-connexin 43, for example, compound is an oligonucleotide and said transdermal penetration agent promotes the delivery of oligonucleotides through the skin.

According to a further aspect of the present invention, provided is a method for reducing pain in a supporting body structure of a subject, which comprises applying to the subject in need thereof a transdermal delivery device comprising, for example, an anti-connexin 43 compound to an area of skin proximal to a site of tissue or joint pain in said subject. Suitably, the anti-connexin 43 compound, for example, is an oligonucleotide and the transdermal delivery device promotes delivery of oligonucleotides through the skin.

One suitable transdermal delivery device is a transdermal microprojection delivery device. Said microprojection device may optionally have a biocompatible coating being formed from a coating formulation having, for example, a anti-connexin 43 compound disposed thereon. An alternate suitable transdermal delivery device is one that forms at least one micropore in a tissue membrane whereby delivery of said anti-connexin 43, for example, compound through the skin is promoted.

In a further aspect, provided is an article of manufacture comprising a packaging material and a transdermal delivery composition contained within said packaging material, wherein said transdermal delivery composition comprises a pain relief effective amount of for example, an anti-connexin 43 compound and a transdermal penetration effective amount of an ethoxylated oil; and wherein said packaging material comprises a label that indicates that said composition may be used for reducing pain in a supporting structure. The article of manufacture may comprise, an ethoxylated oil is selected from the group comprising of ethoxylated macadamia nut oil, ethoxylated meadowfoam oil, ethoxylated castor oil, ethoxylated jojoba oil, ethoxylated corn oil, ethoxylated sunflower oil, ethoxylated sesame oil, and ethoxylated emu oil. Optionally, said anti-connexin 43 compound, for example, is an oligonucleotide.

In another aspect, provided is an article of manufacture comprising a packaging material and a transdermal delivery composition contained within said packaging material, wherein said transdermal delivery composition comprises a pain relief effective amount of, for example, an anti-connexin 43 compound and a transdermal penetration effective amount of an oil; and wherein said packaging material comprises a label that indicates that said composition may be used for reducing pain in a supporting structure. The article of manufacture may comprise an oil selected from the group comprising of macadamia nut oil, meadowfoam oil, castor oil, jojoba oil, corn oil, sunflower oil, sesame oil, and emu oil. Optionally, said anti-connexin 43, for example, compound is an oligonucleotide.

In a further aspect, a method for reducing pain in a supporting body structure of a subject is provided, comprising topically administering to said subject in need thereof a therapeutically effective amount of, for example, a connexin 43 gap junction modulation agent containing transdermal, injectable, instillation, or depot dosage form, whereby pain is reduced.

These and other aspects of the present inventions, which are not limited to or by the information in this Brief Summary, are provided below.

### DETAILED DESCRIPTION

As used herein, a “disorder” is any disorder, disease, or condition involving pain that would benefit from a gap junction modulation agent, including, for example, one or more anti-connexin compounds, gap junction modifying compounds, connexin binding compounds, or hemichannel modulation compounds.

As used herein, “subject” refers to any mammal, including humans, domestic and farm animals, and zoo, sports, or pet animals, such as dogs, horses, cats, sheep, pigs, cows, etc. Non-limiting preferred mammals are a human, including adults, children, and the elderly. Non-limiting preferred sports animals are horses and dogs. Non-limiting preferred pet animals are dogs and cats.

As used herein, “supporting body structure of a subject” refers to joints, muscles, tendons, ligaments, cartilage, and skin of that subject. Particularly useful applications of the present invention include the prevention or treatment of pain in and around joints, including shoulders, hips, ankles, knees, elbows, hands, feet and fingers. Other particularly useful applications of the present invention include the prevention or treatment of pain in the back, particularly the lower back. Each of these may be treated separately, as may each of joints, muscles, tendons, ligaments, cartilage, and skin be the subject of separate treatment for pain.

As used herein, “musculoskeletal system” (also known as the locomotor system) refers to the system that gives animals the ability to physically move using the muscles and the skeletal system. The musculoskeletal system includes the skeleton, made by bones attached to other bones with joints and ligaments, and skeletal muscle attached to the skeleton by tendons. Particularly useful applications of the present invention include the prevention or treatment of musculoskeletal pain, including pain that affects the muscles, ligaments and tendons, along with bones.

As used herein, “pain” includes acute pain and chronic pain. Also included is nerve pain.

As used herein, “preventing” or “prevention” means preventing in whole or in part, or ameliorating, reducing or controlling.

As used herein, a “therapeutically effective amount” or “effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve preventing pain.

As used herein, the terms “treating” and “treatment” refer to both therapeutic treatment and prophylactic or preventative measures.

As used herein, “gap junction modulation agents” are compounds that affect or modulate the activity, properties, expression or formation of a connexin, a connexin hemichannel (connexon), or a gap junction. Gap junction modulation agents include, without limitation, antisense compounds (e.g. antisense polynucleotides), RNAi and siRNA compounds, antibodies and binding fragments thereof, and peptides and polypeptides, which include “peptidomimetics,” and peptide analogs. In addition to anti-connexin polynucleotides and anti-connexin peptides and peptidomimetics, other gap junction modulation agents include compounds that block, inhibit or reduce gap junction opening, including agents that serve to close gap junctions (e.g., connexin phosphorylation compounds), compounds that block, inhibit or reduce hemichannel opening (e.g., connexin phosphorylation compounds), and compounds that block, inhibit or reduce or disrupt ZO-1 protein interactions with connexins (e.g., carboxy-terminal connexin 43 polypeptides). Such gap junction modulation agents are useful for treating a subject to provide pain relief, including relief from pain as a result of trauma, as a result of an orthopedic procedure or surgery, or as a result of an orthopedic disease, disorders and/or condition. Non-limiting preferred gap junction modulation agents are anti-connexin 43 agents, anti-connexin 43 gap junction agents, and anti-connexin 43 hemichannel agents. Exemplary anti-connexin agents are discussed in further detail herein. Other non-limiting preferred gap junction modulation agents are anti-connexin 26 agents, anti-connexin 26 gap junction agents, and anti-connexin 26 hemichannel agents. Non-limiting preferred gap junction modulation agents are anti-connexin 30 agents, anti-connexin 30 gap junction agents, and anti-connexin 30 hemichannel agents.

The terms “peptidomimetic” and “mimetic” include naturally occurring and synthetic chemical compounds that may have substantially the same structural and functional characteristics of protein regions, which they mimic. In the case of connexins, these may mimic, for example, the extracellular loops of opposing connexins involved in connexon-connexon docking and cell-cell channel formation.

“Peptide analogs” refer to the compounds with properties analogous to those of the template peptide and may be non-peptide drugs. “Peptidomimetics” (also known as “mimetic peptides”), which include peptide-based compounds, also include such non-peptide based compounds such as peptide analogs. Peptidomimetics that are structurally similar to therapeutically useful peptides may be used to produce an equivalent or enhanced therapeutic or prophylactic effect. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. The mimetic can be either entirely composed of natural amino acids, or non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. For example, a mimetic composition may be useful as an anti-connexin agent if it is capable of down-regulating biological actions or activities of connexins proteins or hemichannels, such as, for example, preventing the docking of hemichannels to form gap-junction-mediated cell-cell communications, or preventing the opening of hemichannels to expose the cell cytoplasm to the extracellular milieu. Peptidomimetics, mimetic peptides, and connexin modulating peptides, as well as compounds, including connexin phosphorylation compounds and connexin carboxy-terminal polypeptides, encompass those described or referenced herein, as well as those as may be known in the art, whether now known or later developed.

Gap junction modulation agents, include agents that close or block gap junctions and/or hemichannels or otherwise prevent or decrease cell to cell communication via gap junctions or prevent or decrease cell communication to the extracellular environment via hemichannels.

The terms “modulator” and “modulation” of connexin activity, as used herein in its various forms, refers to inhibition in whole or in part of the expression or action or activity of a connexin or connexin hemichannel or connexin gap junction and may function as anti-connexin compounds.

In general, the term “protein” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the teens “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

The term “transdermal”, as used herein, means the delivery of an agent into and/or through the skin for therapy.

The term “transdermal flux”, as used herein, means the rate of transdermal delivery.

As used herein, “transdermal flux rate” is the rate of passage of any analyte out through the skin of an individual, human or animal, or the rate of passage of any permeant, drug, pharmacologically active agent, dye, or pigment in and through the skin of an organism.

The terms “microprojections” and “microprotrusions”, as used herein, refer to piercing elements which are adapted to pierce or cut through the stratum corneum into the underlying epidermis layer, or epidermis and dermis layers, of the skin of a living animal, particularly a mammal and more particularly a human.

The term “microprojection member”, as used herein, generally connotes a microprojection array comprising a plurality of microprojections, often arranged in an array, for piercing the stratum corneum. The microprojection member can be formed in various ways including, for example, by etching or punching a plurality of microprojections from a thin sheet and folding or bending the microprojections out of the plane of the sheet to form a configuration, a three dimensional. The microprojection member can also be formed in other known manners, such as by forming one or more strips having microprojections along an edge of each of the strip(s) as disclosed in U.S. Pat. No. 6,050,988.

The term “coating formulation”, as used herein, is meant to mean and include a composition or mixture that is employed to coat the microprojections and/or arrays thereof. Preferably, the coating formulation includes at least one gap junction modulation agent, which can, for example, be in solution or suspension in the formulation.

The term “biocompatible coating” and “solid coating”, as used herein, is meant to mean and include a “coating formulation” in a substantially solid state.

As used herein, “artificial opening” or “micropore” means any physical breach of the biological membrane of a suitable size for delivering or extraction of a fluid or other composition therethrough, including micropores. “Artificial opening” or “micropore” or any such similar term thus refers to a small hole, opening or crevice created to a desired depth in or through a biological membrane. The opening may be formed via the conduction of thermal energy as described in U.S. Pat. No. 5,885,211, or through a mechanical process, or through a pyrotechnic process, for example. The size of the hole or pore is, for example, approximately 1-1000 microns in diameter. It is to be understood that the term micropore is used in the singular form for simplicity, but that the devices and methods may form multiple openings or pores.

“Iontophoresis” refers to the application of an external electric field to the tissue surface through the use of two or more electrodes and delivery of an ionized form of drug or an un-ionized drug carried with the water flux associated with ion transport (electro-osmosis) into the tissue or the similar extraction of a biological fluid or analyte.

“Electroporation” refers to the creation through electric current flow of openings in cell walls, generally openings that are orders of magnitude smaller than micropores. The openings formed with electroporation are typically only a few nanometers in any dimension. Electroporation is useful to facilitate cellular uptake of selected permeants by the targeted tissues beneath the outer layers of an organism after the permeant has passed through the micropores into these deeper layers of tissue.

“Sonophoresis” or “sonification” refers to sonic energy, which may include frequencies normally described as ultrasonic, generated by vibrating a piezoelectric crystal or other electromechanical element by passing an alternating current through the material. The use of sonic energy to increase the permeability of the skin to drug molecules has been termed sonophoresis or phonophoresis.

“Integrated device” means a device suitable for forming artificial openings in tissue and further suitable for one or more additional applications, for example, delivering one or more permeants into the tissue (preferably through the artificial openings), and optionally collecting a biological fluid from the tissue (preferably through the artificial openings) and optionally analyzing the biological fluid to determine a characteristic thereof.

As used herein, “non-invasive” means not requiring the entry of a needle, catheter, or other invasive medical instrument into apart of the body.

As used herein, “minimally invasive” refers to the use of mechanical, hydraulic, or electrical means that invade the stratum corneum to create a small hole or micropore without causing substantial damage to the underlying tissues.

As used herein, “pharmaceutically acceptable carrier” refers to a carrier in which a substance such as a pharmaceutically acceptable drug could be provided for deliver. Pharmaceutically acceptable carriers are described in the art, for example, in “Remington: The Science and Practice of Pharmacy,” Mack Publishing Company, Pennsylvania, 1995, the disclosure of which is incorporated herein by reference. Carriers could include, for example, water and other aqueous solutions, oils, lipids, saccharides, polysaccharides, buffers, excipients, and biodegradable polymers such as polyesters, polyanhydrides, polyamino acids, liposomes and mixtures thereof.

### General Aspects

In the following disclosure, several transdermal delivery systems are described that can administer an effective amount of a pharmaceutical or cosmetic agent to the human body. Although embodiments of the invention can be used to administer low or high (or both low and high) molecular weight gap junction modulation agents, particularly suitable embodiments include transdermal delivery systems that can administer compounds having molecular weights greater than about 5,000 or 6,000 daltons. One embodiment, for example, includes a transdermal delivery system that can administer a therapeutically effective amount of a gap junction modulation agent for pain relief. Some of these embodiments concern transdermal delivery systems that can administer gap junction modulation agents, such as nucleic acids, peptides and peptidomimetics as well as other gap junction modulation agents. These examples are provided to demonstrate that embodiments of the invention can be used to transdermally deliver both low and high molecular weight compounds and it should be understood that many other molecules can be effectively delivered to the body, using the embodiments described herein, in therapeutically or prophylactically beneficial amounts.

The embodied transdermal delivery formulations described herein may comprise a penetration enhancer that includes a lipid or an ethoxylated lipid. Lipids (e.g., oils) and ethoxylated lipids (e.g., ethoxylated oils) can be used as transdermal penetration vehicles or enhancers to transport low and high molecular weight compounds through the skin. It is also contemplated that ethoxylated fatty acids (e.g., palmitoleic acid or oleic acid) can be used in some embodiments (e.g., in addition to supplement an oil or ethoxylated oil such as an emu oil or macadamia nut oil, or an ethoxylated emu oil or an ethoxylated macadamia nut oil).

An ethoxylated lipid can be created in a number of ways known in the art. An approach useful in conjunction with the transdermal methods of the present invention involves the reaction of ethylene oxide with a vegetable, nut (e.g., macadamia nut), animal (such as emu oil), or synthetic oil. The hydrophilic component of a penetration enhancer can be by virtue of the number of ethoxylations present on the lipid molecule. Additionally, an alcohol, a nonionic solubilizer or an emulsifier may be added to improve the solubility of the delivered agent or effectiveness or fluidity of the penetration enhancer. Suitable hydrophilic components include, but are not limited to, ethylene glycol, propylene glycol, dimethyl sulfoxide (DMSO), dimethyl polysiloxane (DMPX), oleic acid, caprylic acid, isopropyl alcohol, 1-octanol, ethanol (denatured or anhydrous), and other pharmaceutical grade or absolute alcohols.

Embodiments of the invention can also comprise conventionally used agents in the formulation art such as an aqueous adjuvant. Thus, several embodiments of the invention may have a penetration enhancer that includes a hydrophobic/hydrophilic component comprising an ethoxylated oil (e.g., macadamia nut oil, coconut oil, eucalyptus oil, synthetic oils, castor oil, glycerol, corn oil, jojoba oil, or emu oil) and may contain a hydrophilic component comprising an alcohol, a nonionic solubilizer, or an emulsifier (e.g., isopropyl alcohol) and/or, optionally, an aqueous adjuvant.

Other conventional components in a formulation may be used in transdermal delivery formulations of the invention including fragrance, creams, ointments, colorings, and other compounds so long as the added component does not deleteriously affect transdermal delivery of the gap junction modulation agent.

Other examples of transdermal delivery systems useful in the invention include transdermal delivery devices, for example, microporation devices, electroporation devices, iontophoresis devices, sonophoresis devices and microprojection devices and arrays.

Methods of treating and preventing pain are provided. In some embodiments, a transdermal delivery system comprising one or more gap junction modulation agents are provided to a patient in need of treatment, such as for relief of pain. A patient can be contacted with the transdermal delivery system and treatment continued for a time sufficient to reduce pain or prevent pain.

### Gap Junction Modulation Agents

Gap junction modulation agents of the invention described herein are capable of modulating or affecting the transport of molecules into and out of cells (e.g., blocking, reducing, inhibiting or downregulating). Thus, certain gap junction modulation agents described herein modulate cellular communication (e.g., cell to cell). Certain gap junction modulation agents modulate or effect transmission of molecules between the cell cytoplasm and the periplasmic or extracellular space. Such gap junction modulation agents are generally targeted to connexins and/or connexin hemichannels (connexons). Hemichannels and resulting gap junctions that comprise connexins are independently involved in the release or exchange of small molecules between the cell cytoplasm and an extracellular space or tissue in the case of open hemichannels, and between the cytoplasm of adjoining cell in the case of open gap junctions. Thus, an gap junction modulation agent provided herein may directly or indirectly reduce coupling and communication between cells or reduce or block communication (or the transmission of molecules) between a cell and extracellular space or tissue, and the modulation of transport of molecules from a cell into an extracellular space or tissue (or from an extracellular space or tissue into a cell) or between adjoining cells is within the scope of anti-connexin agents and embodiments of the invention. Preferably, the connexin is connexin 43.

Any gap junction modulation agent that is capable of eliciting a desired inhibition of the passage (e.g. transport) of molecules through a gap junction or connexin hemichannel may be used in embodiments of the invention. Any gap junction modulation agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such gap junction modulation agents may modulate the passage of molecules through a gap junction or connexin hemichannel with or without gap junction uncoupling (blocking the transport of molecules through gap junctions). Such compounds include, for example, proteins and polypeptides, polynucleotides, and other organic compounds, and they may, for example block the function or expression of a gap junction or a hemichannel in whole or in part, or downregulate the production of a connexin in whole or in part. Certain gap junction inhibitors are listed in Evans, W. H. and Boitano, S. Biochem. Soc. Trans. 29: 606-612 (2001). Other gap junction modulation agents include connexin phosphorylation compounds that close gap junctions and/or hemichannels, in whole or in part, and connexin carboxy-terminal polypeptides that can inhibit, reduce or block ZO-1 protein binding. Preferably, the connexin is connexin 43, the hemichannel is a connexin 43 hemichannel, and the gap junction is a connexin 43 gap junction.

Certain gap junction modulation agents provide downregulation of connexin expression (for example, by downregulation of mRNA transcription or translation) or otherwise decrease or inhibit the activity of a connexin protein, a connexin hemichannel or a gap junction. In the case of downregulation, this will have the effect of reducing direct cell-cell communication by gap junctions, or exposure of cell cytoplasm to the extracellular space by hemichannels, at the site at which connexin expression is downregulated. Anti-connexin 43 agents are preferred. Other presently preferred embodiments are anti-connexin 26 and anti-connexin 30 agents.

Examples of gap junction modulation agents include agents that decrease or inhibit expression or function of connexin mRNA and/or protein or that decrease activity, expression or formation of a connexin, a connexin hemichannel or a gap junction. Anti-connexin agents include anti-connexin polynucleotides, such as antisense polynucleotides and other polynucleotides (such as polynucleotides having siRNA or ribozyme functionalities), as well as antibodies and binding fragments thereof, and peptides and polypeptides, including peptidomimetics and peptide analogs that modulate hemichannel or gap junction activity or function. Anti-connexin 43 agents are preferred. Other presently preferred embodiments are anti-connexin 26 and anti-connexin 30 agents.

### Polynucleotides

Polynucleotides useful in the invention include connexin antisense polynucleotides as well as polynucleotides which have functionalities which enable them to downregulate connexin expression. Other suitable anti-connexin polynucleotides include RNAi polynucleotides and siRNA polynucleotides. Anti-connexin 43 polynucleotides are preferred. Other presently preferred embodiments are anti-connexin 26 and anti-connexin 30 agents

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss). Methods of synthesizing antibodies and binding fragments as well as peptides and polypeptides, including peptidomimetics and peptide analogs are known to those of skill in the art. See e.g. Lihu Yang et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA, preferably, for example, connexin 43 mRNA. Other presently preferred embodiments are anti-connexin 26 and anti-connexin 30 antisense compounds. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 8. The connexin is typically an a or 13 connexin. Preferably the connexin is an a connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted. Other presently preferred connexin targets are connexin 26 and connexin 30 agents.

Anti-connexin polynucleotides include connexin antisense polynucleotides as well as polynucleotides which have functionalities which enable them to downregulate connexin expression. Other suitable anti-connexin polynucleotides include RNAi polynucleotides and siRNA polynucleotides.

In one non-limiting preferred aspect, the antisense polynucleotides are targeted to the mRNA of one connexin protein only. Most preferably, this connexin protein is connexin 43. In another aspect, the connexin protein is connexin 26 or connexin 30. In another aspect, the connexin protein is connexin 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination. Preferred target connexins in addition to connexin 43 are connexins 26 an 30.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26 and 30. Other connexin proteins to which oligodeoxynucleotides are directed may include, for example, connexins 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable. 15 to 25 mers are also suitable, as were 18 to 22 mers, for example.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ. ID. NO: 1) |
| 5′ GTA ATT GCG GCA GGA GGA ATT GTT TCT GTC 3′ | (connexin 43) | (SEQ. ID. NO: 2) |
| 5′ GGC AAG AGA CAC CAA AGA CAC TAC CAG CAT 3′ | (connexin 43) | (SEQ. ID. NO: 3) |
| 5′ TCC TGA GCA ATA CCT AAC GAA CAA ATA 3′ | (connexin 26) | (SEQ. ID. NO: 4) |
| 5′ CAT CTC CTT GGT GCT CAA CC 3′ | (comiexin 37) | (SEQ. ID. NO: 5) |
| 5′ CTG AAG TCG ACT TGG CTT GG 3′ | (connexin 37) | (SEQ. ID. NO: 6) |
| 5′ CTC AGA TAG TGG CCA GAA TGC 3′ | (connexin 30) | (SEQ. ID. NO: 7) |
| 5′ TTG TCC AGG TGA CTC CAA GG 3′ | (connexin 30) | (SEQ. ID. NO: 8) |
| 5′ CGT CCG AGC CCA GAA AGA TGA GGT C 3′ | (connexin 31.1) | (SEQ. ID. NO: 9) |
| 5′ AGA GGC GCA CGT GAG ACA C 3′ | (connexin 31.1) | (SEQ. ID. NO: 10) |
| 5′ TGA AGA CAA TGA AGA TGT T 3′ | (connexin 31.1) | (SEQ. ID. NO: 11) |
| 5′ TTT CTT TTC TAT GTG CTG TTG GTG A 3′ | (connexin 32) | (SEQ. ID. NO: 12) |

Suitable polynucleotides for the preparation of the combined polynucleotide compositions described herein include for example, polynucleotides to Connexin Cx43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable: GTA ATT GCG GCA AGA AGA ATT GTT TCT GTC (SEQ.ID.NO:1); GTA ATT GCG GCA GGA GGA ATT GTT TCT GTC (SEQ.ID.NO:2); and GGC AAG AGA CAC CAA AGA CAC TAC CAG CAT (SEQ.ID.NO:3).

For example, suitable antisense polynucleotides for connexins 26, 31.1 and 32 have the following sequences:

(SEQ. ID. NO: 4)
5′ TCC TGA GCA ATA CCT AAC GAA CAA ATA (connexin
26);
(SEQ. ID. NO: 9)
5′ CGT CCG AGC CCA GAA AGA TGA GGT C (connexin
31.1);
and
(SEQ. ID. NO: 12)
5′ TTT CTT TTC TAT GTG CTG TTG GTG A (connexin
32).

Other connexin antisense polynucleotide sequences useful according to the methods of the present invention include:

(SEQ. ID. NO: 5)
5′ CAT CTC CTT GGT GCT CAA CC 3′ (connexin 37);
(SEQ. ID. NO: 6)
5′ CTG AAG TCG ACT TGG CTT GG 3′ (connexin 37);
(SEQ. ID. NO: 7)
5′ CTC AGA TAG TGG CCA GAA TGC 3′ (connexin 30);
(SEQ. ID. NO: 8)
5′ TTG TCC AGG TGA CTC CAA GG 3′ (connexin 30);
(SEQ. ID. NO: 10)
5′ AGA GGC GCA CGT GAG ACA C 3′ (connexin 31.1);
and
(SEQ. ID. NO: 11)
5′ TGA AGA CAA TGA AGA TGT T 3′ (connexin 31.1).

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

### Polynucleotide Homologues

Anti-connexin polynucleotides include anti-connexin polynucleotide homologues. Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program, which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Agents

Binding proteins, including peptides, peptidomimetics, antibodies, antibody fragments, and the like, are also suitable modulators of gap junctions and hemichannels.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. J Cell Biol 107: 1817-1824 (1988); Meyer R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). Short peptides corresponding to sequences within the extracellular loops of connexins were said to inhibit intercellular communication. Boitano S. and Evans W. Am J Physiol Lung Cell Mol Physiol 279: L623-L630 (2000). The use of peptides as inhibitors of cell-cell channel formation produced by connexin (Cx) 32 expressed in paired Xenopus oocytes has also been reported. Dahl G, et al., Biophys J 67: 1816-1822 (1994). Berthoud, V. M. and Seul, K. H., summarized some of these results. Am J., Physiol. Lung Cell Mol. Physiol. 279: L619-L622 (2000).

Anti-connexin agents include peptides comprising an amino acid sequence corresponding to a transmembrane region (e.g. 1^{st} to 4^{th}) of a connexin (e.g. connexin 45, 43, 26, 30, 31.1, and 37). Anti-connexin agents may comprise a peptide comprising an amino acid sequence corresponding to a portion of a transmembrane region of a connexin 45. Anti-connexin agents include a peptide having an amino acid sequence that comprises about 5 to 20 contiguous amino acids of SEQ.ID.NO:13, a peptide having an amino acid sequence that comprises about 8 to 15 contiguous amino acids of SEQ.ID.NO:13, or a peptide having an amino acid sequence that comprises about 11 to 13 contiguous amino acids of SEQ.ID.NO:13. Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:13. In certain anti-connexin agents provided herein, the extracellular domains of connexin 45 corresponding to the amino acids at positions 46-75 and 199-228 of SEQ ID NO: 13 may be used to develop the particular peptide sequences. Certain peptides described herein have an amino acid sequence corresponding to the regions at positions 46-75 and 199-228 of SEQ.ID.NO: 13. The peptides need not have an amino acid sequence identical to those portions of SEQ.ID.NO: 13, and conservative amino acid changes may be made such that the peptides retain binding activity or functional activity. Alternatively, the peptide may target regions of the connexin protein other than the extracellular domains (e.g. the portions of SEQ.ID.NO:13 not corresponding to positions 46-75 and 199-228).

Also, suitable anti-connexin agents comprise a peptide comprising an amino acid sequence corresponding to a portion of a transmembrane region of a connexin 43. Anti-connexin agents include peptides having an amino acid sequence that comprises about 5 to 20 contiguous amino acids of SEQ.ID.NO:14, peptides having an amino acid sequence that comprises about 8 to 15 contiguous amino acids of SEQ.ID.NO:14, or peptides having an amino acid sequence that comprises about 11 to 13 contiguous amino acids of SEQ.ID.NO:14. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:14. Other anti-connexin agents comprise the extracellular domains of connexin 43 corresponding to the amino acids at positions 37-76 and 178-208 of SEQ.ID.NO: 14. Anti-connexin agents include peptides described herein which have an amino acid sequence corresponding to the regions at positions 37-76 and 178-208 of SEQ.ID.NO: 14. The peptides need not have an amino acid sequence identical to those portions of SEQ.ID.NO: 14, and conservative amino acid changes may be made such that the peptides retain binding activity or functional activity. Alternatively, peptides may target regions of the connexin protein other than the extracellular domains (e.g. the portions of SEQ.ID.NO:14 not corresponding to positions 37-76 and 178-208).

```
Connexin 45
(SEQ ID NO. 13)
Met Ser Trp Ser Phe Leu Thr Arg Leu Leu Glu Glu Ile His Asn His
1 5 10 15
Ser Thr Phe Val Gly Lys Ile Trp Leu Thr Val Leu Ile Val Phe Arg
                  20 25 30
Ile Val Leu Thr Ala Val Gly Gly Glu Ser Ile Tyr Tyr Asp Glu Gln
                35 40 45
Ser Lys Phe Val Cys Asn Thr Glu Gln Pro Gly Cys Glu Asn Val Cys
        50 55 60
Tyr Asp Ala Phe Ala Pro Leu Ser His Val Arg Phe Trp Val Phe Gln
65 70 75 80
Ile Ile Leu Val Ala Thr Pro Ser Val Met Tyr Leu Gly Tyr Ala Ile
                                85 90 95
His Lys Ile Ala Lys Met Glu His Gly Glu Ala Asp Lys Lys Ala Ala
                        100 105 110
Arg Ser Lys Pro Tyr Ala Met Arg Trp Lys Gln His Arg Ala Leu Glu
                115 120 125
Glu Thr Glu Glu Asp Asn Glu Glu Asp Pro Met Met Tyr Pro Glu Met
        130 135 140
Glu Leu Glu Ser Asp Lys Glu Asn Lys Glu Gln Ser Gln Pro Lys Pro
145 150 155 160
Lys His Asp Gly Arg Arg Arg Ile Arg Glu Asp Gly Leu Met Lys Ile
                                165 170 175
Tyr Val Leu Gln Leu Leu Ala Arg Thr Val Phe Glu Val Gly Phe Leu
                        180 185 190
Ile Gly Gln Tyr Phe Leu Tyr Gly Phe Gln Val His Pro Phe Tyr Val
                195 200 205
Cys Ser Arg Leu Pro Cys Pro His Lys Ile Asp Cys Phe Ile Ser Arg
        210 215 220
Pro Thr Glu Lys Thr Ile Phe Leu Leu Ile Met Tyr Gly Val Thr Gly
225 230 235 240
Leu Cys Leu Leu Leu Asn Ile Trp Glu Met Leu His Leu Gly Phe Gly
                                245 250 255
Thr Ile Arg Asp Ser Leu Asn Ser Lys Arg Arg Glu Leu Glu Asp Pro
                        260 265 270
Gly Ala Tyr Asn Tyr Pro Phe Thr Trp Asn Thr Pro Ser Ala Pro Pro
                275 280 285
Gly Tyr Asn Ile Ala Val Lys Pro Asp Gln Ile Gln Tyr Thr Glu Leu
        290 295 300
Ser Asn Ala Lys Ile Ala Tyr Lys Gln Asn Lys Ala Asn Thr Ala Gln
305 310 315 320
Glu Gln Gln Tyr Gly Ser His Glu Glu Asn Leu Pro Ala Asp Leu Glu
                                325 330 335
Ala Leu Gln Arg Glu Ile Arg Met Ala Gln Glu Arg Leu Asp Leu Ala
                        340 345 350
Val Gln Ala Tyr Ser His Gln Asn Asn Pro His Gly Pro Arg Glu Lys
                355 360 365
Lys Ala Lys Val Gly Ser Lys Ala Gly Ser Asn Lys Ser Thr Ala Ser
        370 375 380
Ser Lys Ser Gly Asp Gly Lys Asn Ser Val Trp Ile
385 390 395
Connexin 43
(SEQ ID NO. 14)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Glu Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                        180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

The anti-connexin peptides may comprise sequences corresponding to a portion of the connexin extracellular domains with conservative amino acid substitutions such that peptides are functionally active anti-connexin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 are shown below in Table 2. M1, 2, 3 and 4 refer to the 1^{st} to 4^{th} transmembrane regions of the connexin 43 protein respectively. E1 and E2 refer to the first and second extracellular loops respectively.

TABLE 2
Peptidic Inhibitors of Intercellular Communication
(cx43)

| | | |
|---|---|---|
| FEVAFLLIQWI | M3 & E2 | (SEQ. ID. NO: 15) |
| LLIQWYIGFSL | E2 | (SEQ. ID. NO: 16) |
| SLSAVYTCKRDPCPHQ | E2 | (SEQ. ID. NO: 17) |
| VDCFLSRPTEKT | E2 | (SEQ. ID. NO: 18) |
| SRPTEKTIFII | E2 & M4 | (SEQ. ID. NO: 19) |
| LGTAVESAWGDEQ | M1 & E1 | (SEQ. ID. NO: 20) |
| QSAFRCNTQQPG | E1 | (SEQ. ID. NO: 21) |
| QQPGCENVCYDK | E1 | (SEQ. ID. NO: 22) |
| VCYDKSFPISHVR | E1 | (SEQ. ID. NO: 23) |

Table 3 provides additional exemplary connexin peptides used in inhibiting hemichannel or gap junction function. In other embodiments, conservative amino acid changes are made to the peptides or fragments thereof.

TABLE 3
Additional Peptidic Inhibitors of Intercellular
Communication (cx32, cx43)

| | | | AA's and |
|---|---|---|---|
| Connexin | Location | | Sequence |
| Cx32 | El 39-77 | AAESVWGDEIKSSFICNTLQP | (SEQ. ID. |
| | | GCNSVCYDHFFPISHVR | NO: 24) |
| Cx32 | E1 41-52 | ESVWGDEKSSFI | (SEQ. ID. |
| | | | NO: 25) |
| Cx32 | E1 52-63 | ICNTLQPGCNSV | (SEQ. ID. |
| | | | NO: 26) |
| Cx32 | E1 62-73 | SVCYDHFFPISII | (SEQ. ID. |
| | | | NO: 27) |
| Cx32 | E2 | RLVKCEAFPCPNTVDCFVSRP | (SEQ. ID. |
| | 64-188 | TEKT | NO: 28) |
| Cx32 | E2 | VKCEAFPCPNTV | (SEQ. ID. |
| | 166-177 | | NO: 29) |
| Cx32 | E2 | VDCFVSRPTEKT | (SEQ. ID. |
| | 177-188 | | NO: 30) |
| Cx32 | E1 63-75 | VCYDHFFPISHVR | (SEQ. ID. |
| | | | NO: 31) |
| Cx32 | E1 45-59 | VWGDEKSSFICNTLQPGY | (SEQ. ID. |
| | | | NO: 32) |
| Cx32 | E1 46-59 | DEKSSFICNTLQPGY | (SEQ. ID. |
| | | | NO: 33) |
| Cx32 | E2 | SRPTEKTVFTV | (SEQ. ID. |
| | 182-192 | | NO: 34) |
| Cx32/Cx43 | E2 182- | SRPTEKT | (SEQ. ID. |
| | 188/201- | | NO: 35) |
| | 207 | | |
| Cx32 | E1 52-63 | ICNTLQPGCNSV | (SEQ. ID. |
| | | | NO: 36) |
| Cx40 | E2 | FLDTLHVCRRSPCPHP | (SEQ. ID. |
| | 177-192 | | NO: 37) |
| Cx43 | E2 | KRDPCHQVDCFLSRPTEK | (SEQ. ID. |
| | 188-205 | | NO: 38) |

Table 4 provides the extracellular loops for connexin family members which are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 4
Extracellular loops for various cormexin family members
E1

| | | |
|---|---|---|
| huCx26 | KEVWGDEQADFVCNTLQPGCKNVCYDHYFPISHIR | (SEQ. ID. NO: 39) |
| huCx30 | QEVWGDEQEDFVCNTLQPGCKNVCYDHFFPVSHIR | (SEQ. ID. NO: 40) |
| huCx30.3 | EEVWDDEQKDFVCNTKQPGCPNVCYDEFFPVSHVR | (SEQ. ID. NO: 41) |
| huCx31 | ERVWGDEQKDFDCNTKQPGCTNVCYDNYFPISNIR | (SEQ. ID. NO: 42) |
| huCx31.1 | ERVWSDDHKDFDCNTRQPGCSNVCFDEFFPVSHVR | (SEQ. ID. NO: 43) |
| huCx32 | ESVWGDEKSSFICNTLQPGCNSVCYDQFFPISHVR | (SEQ. ID. NO: 44) |
| huCx36 | ESVWGDEQSDFECNTAQPGCTNVCYDQAFPISHIR | (SEQ. ID. NO: 45) |
| huCx37 | ESVWGDEQSDFECNTAQPGCTNVCYDQAFPISHIR | (SEQ. ID. NO: 46) |
| huCx40.1 | RPVYQDEQERFVCNTLQPGCANVCYDVFSPVSHLR | (SEQ. ID. NO: 47) |
| huCx43 | ESAWGDEQSAFRCNTQQPGCENVCYDKSFPISHVR | (SEQ. ID. NO: 48) |
| huCx46 | EDVWGDEQSDFTCNTQQPGCBNVCYBRAFPISHIR | (SEQ. ID. NO: 49) |
| huCx46.6 | EAIYSDEQAKFTCNTRQPGCDNVCYDAFAPLSHVR | (SEQ. ID. NO: 50) |
| huCx40 | ESSWGDEQADFRCDTIQPGCQNVCTDQAFPISHIR | (SEQ. ID. NO: 51) |
| huCx45 | GESIYYDEQSKFVCNTEQPGCENVCYDAFAPLSHVR | (SEQ. ID. NO: 52) |

E2

| | | |
|---|---|---|
| huCx26 | MYVFYVMYDGFSMQRLVKCNAWPCPNTVDCFVSRPTEKT | (SEQ. ID. NO: 53) |
| huCx30 | MYVFYFLYNGYHLPWVLKCGIDPCPNLVDCFISRPTEKT | (SEQ. ID. NO: 54) |
| huCx30.3 | LYIFHRLYKDYDMPRVVACSVEPCPHTVDCYISRPTEKK | (SEQ. ID. NO: 55) |
| huCx31 | LYLLHTLWHGFNMPRLVQCANVAPCPNIVDCYIARPTEKK | (SEQ. ID. NO: 56) |
| huCx31.1 | LYVFHSFYPKYILPPVVKCHADPCPNIVDCFISKPSEKN | (SEQ. ID. NO: 57) |
| huCx32 | MYVFYLLYPGYAMVRLVKCDVYPCPNTVDCFVSRPTEKT | SEQ. ID. NO: 58) |
| huCx36 | LYGWTMEPVFVCQRAPCPYLVDCFVSRPTEKT | (SEQ. ID. NO: 59) |
| huCx37 | LYGWTMEPVFVCQRAPCPYLVDCFVSRPTEKT | (SEQ. ID. NO: 60) |
| huCx40.1 | GALHYFLFGFLAPKKFPCTRPPCTGVVDCYVSRPTSKS | (SEQ. ID. NO: 61) |
| huCx43 | LLIQWYIYGFSLSAVYTCKRDPCPHQVDCFLSRPTEKT | (SEQ. ID. NO: 62) |
| huCx46 | IAGQYFLYGFELKPLYRCDRWPCPNTVDCFISRPTEKT | (SEQ. ID. NO: 63) |
| huCx46.6 | LVGQYLLYGFEVRPFFPCSRQPCPHVVDCFVSRPTEKT | (SEQ. ID. NO: 64) |
| huCx40 | IVGQYFIYGIFLTTLHVCRRSPCPHPVNCYVSRPTEKN | (SEQ. ID. NO: 65) |
| huCx45 | LIGQYFLYGFQVHPFYVCSRLPCHPKIDCFISRPTEKT | (SEQ. ID. NO: 66) |

Table 5 provides the extracellular domain for connexin family members which may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 5
Extracellular domains

| | | |
|---|---|---|
| Peptide | VDCFLSRPTEKT | (SEQ. ID. NO: 18) |
| Peptide | SRPTEKTIFII | (SEQ. ID. NO: 19) |
| huCx43 | LLIQWYTYGFSLSAVYTCKRDPCPHQVDCFLSRPTEKTIFII | (SEQ. ID. NO: 67) |
| huCx26 | MYVFYVMYDGFSMQRLVKCNAWPCPNTVDCFVSRPTEKTVFTV | (SEQ. ID. NO: 68) |
| huCx30 | YVFYFLYNGYHLPWVLKCGIDPCPNLVDCFISRPTEKTVFTI | (SEQ. ID. NO: 69) |
| huCx30.3 | LYIFHRLYKDYDMPRVVACSVEPCPHTVDCYISRPTEKKVFTY | (SEQ. ID. NO: 70) |
| huCx31 | LYLLHTLWHGFNMPRLVQCANVAPCPNIVDCYIARPTEKKTY | (SEQ. ID. NO: 71) |
| huCx31.1 | LYVFHSFYPKYILPPVVKCHADPCPNIVDCFISKPSEKNIFTL | (SEQ. ID. NO: 72) |
| huCx32 | MYVFYLLYPGYAMVRLVKCDVYPCPNTVDCFVSRPTEKTVFTV | (SEQ. ID. NO: 73) |
| huCx36 | LYGWTMEPVFVCQRAPCPYLVDCFVSRPTEKTIFII | (SEQ. ID. NO: 74) |
| huCx37 | LYGWTMEPVFVCQRAPCPYLVDCFVSRPTEKTIFII | (SEQ. ID. NO: 75) |
| huCx40.1 | GALHYFLFGFLAPKKFPCTRPPCTGVVDCYVSRPTEKSLLML | (SEQ. ID. NO: 76) |
| huCx46 | IAGQYFLYGFELKPLYRCDRWPCPNTVDCFISRPTEKTIFII | (SEQ. ID. NO: 77) |
| huCx46.6 | LVGQYLLYGFEVRPFFPCSRQPCPHVVDCFVSRPTEKTVFLL | (SEQ. ID. NO: 78) |
| huCx40 | IVGQYFIYGIFLTTLHVCRRSPCPHPVNCYSRPTEKNVFIV | (SEQ. ID. NO: 79) |
| huCx45 | LIGQYFLYGFQVIIPFYVCSRLPCHPKIDCFISRPTEKTIFLL | (SEQ. ID. NO: 80) |

Table 6 provides peptides inhibitors of connexin 40 shown with reference to the extracellular loops (E1 and E2) of connexin 40. The bold amino acids are directed to the transmembrane regions of connexin 40.

TABLE 6
Cx40 peptide inhibitors
E2

| | |
|---|---|
| LGTAAESSWGDEQADFRCDTIQPGCQNVCTDQAFPISHIRFWVLQ | (SEQ. ID. NO: 94) |
| LGTAAESSWGDEQA | (SEQ. ID. NO: 94) |
| DEQADFRCDTIQP | (SEQ. ID. NO: 94) |
| TIQPGCQNVCTDQ | (SEQ. ID. NO: 94) |
| VCTDQAFPISHIR | (SEQ. ID. NO: 94) |
| AFPISHIRFWVLQ | (SEQ. ID. NO: 94) |

E2

| | |
|---|---|
| MEVGFIVGQYFIYGIFLTTLHVCRRSPCPHPVNCYVSRPTEKNVFIV | (SEQ. ID. NO: 94) |
| MEVGFIVGQYF | (SEQ. ID. NO: 94) |
| IVGQYFIYGIFL | (SEQ. ID. NO: 94) |
| GIFLTTLHVCRRSP | (SEQ. ID. NO: 94) |
| RRSPCPHPVNCY | (SEQ. ID. NO: 94) |
| VNCYVSRPTEKN | (SEQ. ID. NO: 94) |
| SRPTEKNVFIV | (SEQ. ID. NO: 94) |

Table 7 provides peptides inhibitors of connexin 45 shown with reference to the extracellular loops (E1 and E2) of connexin 45. The bold amino acids are directed to the transmembrane regions of connexin 45.

TABLE 7
Cx45 peptide inhibitors
E1

| | |
|---|---|
| LTAVGGESIYYDEQSKFVCNTEQPGCENVCYDAFAPLSHVRFWVFQ | (SEQ. ID. NO: 94) |
| LTAVGGESIYYDEQS | (SEQ. ID. NO: 95) |
| DEQSKFVCNTEQP | (SEQ. ID. NO: 96) |
| TEQPGCENVCYDA | (SEQ. ID. NO: 97) |
| VCYDAFAPLSHVR | (SEQ. ID. NO: 98) |
| APLSHVRFWVFQ | (SEQ. ID. NO: 99) |

E2

| | |
|---|---|
| FEVGFLIGQYFLYGFQVHPFYVCSRLPCHPKIDCFISRPTEKTIFLL | (SEQ. ID. NO: 100) |
| FEVGFLIGQYF | (SEQ. ID. NO: 101) |
| LIGQYFLYGFQV | (SEQ. ID. NO: 102) |
| GFQVHPFYVCSRLP | (SEQ. ID. NO: 103) |
| SRLPCHPKIDCF | (SEQ. ID. NO: 104) |
| IDCFISRPTEKT | (SEQ. ID. NO: 105) |
| SRPTEKTIFLL | (SEQ. ID. NO: 106) |

In certain embodiments, it is preferred that certain peptide inhibitors block hemichannels without disrupting existing gap junctions. While not wishing to be bound to any particular theory or mechanism, it is also believed that certain peptidomimetics (e.g. VCYDKSFPISHVR, (SEQ.ID.NO: 23) block hemichannels without causing uncoupling of gap junctions (See Leybeart et al., Cell Commun. Adhes. 10: 251-257 (2003)), or do so in lower dose amounts. The peptide SRPTEKTIFII (SEQ.ID.NO: 19) may also be used, for example to block hemichannels without uncoupling of gap junctions. The peptide SRGGEKNVFIV (SEQ.ID.NO: 107) may be used that as a control sequence (DeVriese et al., Kidney Internat. 61: 177-185 (2002)). Examples of peptide inhibitors for connexin 45 YVCSRLPCHP (SEQ.ID.NO:108), QVHPFYVCSRL (SEQ.ID.NO:109), FEVGFLIGQYFLY (SEQ.ID.NO:110), GQYFLYGFQVHP (SEQ.ID.NO:111), GFQVHPFYVCSR (SEQ.ID.NO:112), AVGGESIYYDEQ (SEQ.ID.NO), YDEQSKFVCNTE (SEQ.ID.NO:114), NTEQPGCENVCY (SEQ.ID.NO:115), CYDAFAPLSHVR (SEQ.ID.NO:116), FAPLSHVRFWVF (SEQ.ID.NO:117) and LIGQY (SEQ.ID.NO:118), QVHPF (SEQ.ID.NO:119), YVCSR (SEQ.ID.NO:120), SRLPC (SEQ.ID.NO:121), LPCHP (SEQ.ID.NO:122) and GESIY (SEQ.ID.NO:123), YDEQSK (SEQ.ID.NO:124), SKFVCN (SEQ.ID.NO:125), TEQPGCEN (SEQ.ID.NO:126), VCYDAFAP (SEQ.ID.NO:127), LSHVRFWVFQ (SEQ.ID.NO:128) The peptides may only be 3 amino acids in length, including SRL, PCH, LCP, CHP, IYY, SKF, QPC, VCY, APL, HVR, or longer, for example: LIQYFLYGFQVHPF (SEQ.ID.NO:129), VHPFYCSRLPCHP (SEQ.ID.NO:130), VGGESIYYDEQSKFVCNTEQPG (SEQ.ID.NO:131), TEQPGCENVCYDAFAPLSHVRF (SEQ.ID.NO:132), AFAPLSHVRFWVFQ (SEQ.ID.NO: 133).

TABLE 8
Table 8A
Human Connexin 43 from GenBank Accession No. M65188
(SEQ. ID. NO: 134)

| | |
|---|---|
| 1 | ggcttttagc gtgaggaaag taccaaacag cagcggagtt ttaaacttta aatagacagg |
| 61 | tctgagtgcc tgaacttgcc ttttcatttt acttcatcct ccaaggagtt caatcacttg |
| 121 | gcgtgacttc actactttta agcaaaagag tggtgcccag gcaacatggg tgactggagc |
| 181 | gccttaggca aactccttga caaggttcaa gcctactcaa ctgctggagg gaaggtgtgg |
| 241 | ctgtcagtac ttttcatttt ccgaatcctg ctgctgggga cagcggttga gtcagcctgg |
| 301 | ggagatgagc agtctgcctt tcgttgtaac actcagcaac ctggttgtga aaatgtctgc |
| 361 | tatgacaagt ctttcccaat ctctcatgtg cgcttctggg tcctgcagat catatttgtg |
| 421 | tctgtaccca cactcttgta cctggctcat gtgttctatg tgatgcgaaa ggaagagaaa |
| 481 | ctgaacaaga aagaggaaga actcaaggtt gcccaaactg atggtgtcaa tgtggacatg |
| 541 | cacttgaagc agattgagat aaagaagttc aagtacggta ttgaagagca tggtaaggtg |
| 601 | aaaatgcgag gggggttgct gcgaacctac atcatcagta tcctcttcaa gtctatcttt |
| 661 | gaggtggcct tcttgctgat ccagtggtac atctatggat tcagcttgag tgctgtttac |
| 721 | acttgcaaaa gagatccctg cccacatcag gtggactgtt tcctctctcg ccccacggag |
| 781 | aaaaccatct tcatcatctt catgctggtg gtgtccttgg tgtccctggc cttgaatatc |
| 841 | attgaactct tctatgtttt cttcaagggc gttaaggatc gggttaaggg aaagagcgac |
| 901 | ccttaccatg cgaccagtgg tgcgctgagc cctgccaaag actgtgggtc tcaaaaatat |
| 961 | gcttatttca atggctgctc ctcaccaacc gctcccctct cgcctatgtc tcctcctggg |
| 1021 | tacaagctgg ttactggcga cagaaacaat tcttcttgcc gcaattacaa caagcaagca |
| 1081 | agtgagcaaa actgggctaa ttacagtgca gaacaaaatc gaatggggca ggcgggaagc |
| 1141 | accatctcta actcccatgc acagcctttt gatttccccg atgataacca gaattctaaa |
| 1201 | aaactagctg ctggacatga attacagcca ctagccattg tggaccagcg accttcaagc |
| 1261 | agagccagca gtcgtgccag cagcagacct cggcctgatg acctggagat ctag |

Table 8B
Human Connexin 43
(SEQ. ID. NO: 135)

| | |
|---|---|
| 1 | atgggtgactggagcgcctt aggcaaactc cttgacaagg ttcaagccta ctcaactgct |
| 61 | ggagggaaggtgtggctgtc agtacttttc attttccgaatcctgctgct ggggacagcg |
| 121 | gttgagtcagcctggggaga tgagcagtct gcctttcgtt gtaacactca gcaacctggt |
| 181 | tgtgaaaatg tctgctatga caagtctttcccaatctctc atgtgcgctt ctgggtcctg |
| 241 | cagatcatat ttgtgtctgt acccacactcttgtacctgg ctcatgtgttctatgtgatg |
| 301 | cgaaaggaag agaaactgaa caagaaagag gaagaactca aggttgccca aactgatggt |
| 361 | gtcaatgtgg acatgcactt gaagcagatt gagataaagaagttcaagta cggtattgaa |
| 421 | gagcatggta aggtgaaaat gcgagggggg ttgctgcgaa cctacatcat cagtatcctc |
| 481 | ttcaagtcta tctttgaggt ggccttcttg ctgatccagt ggtacatcta tggattcagc |
| 541 | ttgagtgctg tttacacttg caaaagagat ccctgcccac atcaggtgga ctgtttcctc |
| 601 | tctcgcccca cggagaaaac catcttcatc atcttcatgc tggtggtgtc cttggtgtcc |
| 661 | ctggccttga atatcattga actcttctat gttttcttca agggcgttaa ggatcgggtt |
| 721 | aagggaaaga gcgaccctta ccatgcgacc agtggtgegc tgagccctgc caaagactgt |
| 781 | gggtctcaaa aatatgctta tttcaatggc tgctcctcac caaccgctcc cctctcgcct |
| 841 | atgtetccte ctgggtacaa gctggttact ggcgacagaa acaattcttc ttgccgcaat |
| 901 | tacaacaage aagcaagtga gcaaaactgg gctaattaca gtgcagaaca aaatcgaatg |
| 961 | gggcaggcgg gaagcaccat ctctaactcc catgcacagccttttgattt ccccgatgat |
| 1021 | aaccagaatt ctaaaaaactagctgagga catgaattac agccactagc cattgtggac |
| 1081 | cagcgacctt caagcagagc cagcagtcgtgccagcagca gacctcggcctgatgacctg |
| 1141 | gagatctag |

### Gap Junction Modifying Agents—Other Anti-Connexin Agents

Gap junction modulation agents include agents that close or block gap junctions and/or hemichannels or otherwise prevent or decrease cell to cell communication via gap junctions or prevent or decrease cell communication to the extracellular environment via hemichannels. They include agents or compounds that prevent, decrease or inhibit, in whole or in part, the activity, function, or formation of a hemichannel or a gap junction.

In certain embodiments, a gap junction modulation agent induces closure, in whole or in part, of a hemichannel or a gap junction. In other embodiments, a gap junction modifying agent blocks, in whole or in part, a hemichannel or a gap junction. In certain embodiments, a gap junction modifying agent decreases or prevents, in whole or in part, the opening of a hemichannel or gap junction.

In certain embodiments, said blocking or closure of a gap junction or hemichannel by a gap junction modifying agent can reduce or inhibit extracellular hemichannel communication by preventing or decreasing the flow of small molecules through an open channel to and from an extracellular or periplasmic space.

Gap junction modifying agents used for closing hemichannels or gap junctions (e.g. phosphorylating connexin 43 tyrosine residues) have been reported in U.S. Pat. No. 7,153,822 to Jensen et al., U.S. Pat. No. 7,250,397, and assorted patent publications. Exemplary gap junction modifying agents also include peptides and peptidomimetics, and are reported in Green et al., WO2006134494. See also Gourdie et al., see WO2006069181, and Tudor et al., see WO2003032964 with regard to connexin carboxy-terminal polypeptides that are said to, for example, inhibit ZO-1 protein binding.

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B, J. Biol. Chem., Vol. 276, Issue 11, 8544-8549, Mar. 16, 2001. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie et al., WO2006/069181.

In certain another aspect, gap junction modifying agent may include, for example, aliphatic alcohols; octanol; heptanol; anesthetics (e.g. halothane), ethrane, fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g. arachidonic acid, oleic acid and lipoxygenase metabolites; Fenamates (flufenamic (FFA), niflumic (NFA) and meclofenamic acids (MFA)); Genistein; glycyrrhetinic acid (GA):18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g. acidifying agents; polyunsaturated fatty acids; fatty acid GEC inhibitors (e.g. oleic and arachidonic acids); quinidine; quinine; all trans-retinoic acid; and tamoxifen.

### Methods and Devices for Transdermal Delivery

As used herein, transdermal delivery can be carried out by methods known in the art or later discovered, including, for example, methods directed to 1) the use of chemical penetration enhancers or skin enhancers; 2) liposome-mediated delivery; 3) iontophoresis; 4) electroporation; 5) sonophoresis; 6) mechanical (e.g., microporation) devices. Exemplary methods suitable for transdermal delivery of the agents disclosed herein can include, for example, methods directed to enhancing the transport of material across the skin pores by increasing the rate of transport across existing pores or by amplifying the number of available skin pores through the creation of artificial pores.

For example, in certain embodiments, transdermal delivery can be carried out by the use of chemical or penetration enhancers, including for example, an pharmaceutically acceptable oil of vegetable, nut, synthetic or animal origin including emu oil, ethoxylated oil, PEG, linoleic acid, ethanol, 1-methanol, and/or agents which delipidize the stratum corneum. Suitable oils include meadowfoam oil, castor oil, jojoba oil, corn oil, sunflower oil, sesame oil, and emu oil, all of which may be optionally ethoxylated. Exemplars include those as described in U.S. Pat. No. 7,291,591, U.S. Pat. No. 7,201,919, U.S. Pat. No. 7,052,715, U.S. Pat. No. 7,033,998, U.S. Pat. No. 6,946,144; U.S. Pat. No. 6,951,658, U.S. Pat. No. 6,759,056, U.S. Pat. No. 6,720,001, U.S. Pat. No. 6,224,853; U.S. Pat. No. 5,695,779; and U.S. Pat. No. 6,750,291. In addition, transdermal patches can also be adapted for delivery of dry powder or lyophilized drugs, and exemplars include those described in U.S. Pat. No. 5,983,135.

In certain embodiments, transdermal delivery can be carried out by liposome mediated delivery methods (e.g., delivery facilitated by application of lipophilic membrane active agents). Suitable exemplars may include those described in U.S. Pat. No. 5,910,306, U.S. Pat. No. 5,718,914, and U.S. Pat. No. 5,064,655.

It will be appreciated by one having ordinary skill in the art that in order to facilitate drug transport across the skin barrier, the transdermal delivery systems can also be employed in conjunction with a wide variety of iontophoresis or electrotransport systems, and the invention is not limited in any way in this regard. Illustrative electrotransport drug delivery systems are disclosed in U.S. Pat. Nos. 5,147,296, 5,080,646, 5,169,382 and 5,169383.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may be achieved in various manners.

In certain embodiments, transdermal delivery can be carried out by iontophoretic methods (e.g., delivery facilitated by application of low level electrical field to the skin over time). Suitable exemplars may include those described in U.S. Pat. No. 6,731,987, U.S. Pat. No. 6,391,015, U.S. Pat. No. 6,553,255 B1; U.S. Pat. No. 4,940,456, U.S. Pat. No. 5,681,580 and U.S. Pat. No. 6,248,349.

In certain embodiments, transdermal delivery can be carried out by electroporation methods (e.g., delivery facilitated by brief application of high voltage pulse to create transient pores in the skin). Suitable exemplars may include U.S. Pat. No. 7,008,637, U.S. Pat. No. 6,706,032, U.S. Pat. No. 6,692,456, U.S. Pat. No. 6,587,705, U.S. Pat. No. 6,512,950, U.S. Pat. No. 6,041,253, U.S. Pat. No. 5,968,006 and U.S. Pat. No. 5,749,847.

In certain embodiments, transdermal delivery can be carried out by sonophoresis methods (e.g., delivery facilitated by application of pulses of low frequency ultrasound to increase skin permeability). Suitable exemplars may include U.S. Pat. No. 7,232,431, U.S. Pat. No. 7,004,933, U.S. Pat. No. 6,842,641, U.S. Pat. No. 6,868,286, U.S. Pat. No. 6,712,805, U.S. Pat. No. 6,575,956, U.S. Pat. No. 6,491,657, U.S. Pat. No. 6,487,447, U.S. Pat. No. 6,234,99, and U.S. Pat. No. 6,190,315.

In certain embodiments, transdermal delivery can be carried out by methods comprising the use of mechanical devices and/or creation of artificial micropores or microchannels (e.g., microprojections) by inducing mechanical alterations or disruptions in the structural elements, thermal stability properties, membrane fluidity and integrity of the dermal architecture and substructures. Suitable exemplars may include MicroPor (Altea Therapeutics), MacroFlux (Alza Corporation), as well as those as described in U.S. Pat. No. 6,893,655, U.S. Pat. No. 6,730,318, U.S. RE35474, U.S. Pat. No. 5,484,604, U.S. Pat. No. 5,362,308, U.S. Pat. No. 5,320,850, and U.S. Pat. No. 5,279,544.

U.S. Pat. No. 7,141,034 describes a device and a method for painlessly creating microscopic holes, i.e., micropores, from about 1 to 1000 microns across, in the stratum corneum of human skin. The device uses thermal energy source, or heat probe, which is held in contact with the stratum corneum, for creating micropores. The thermal micropores are created using short time-scale (1 microsecond to 50 milliseconds), thermal energy pulses to ablate the tissue of biological membranes. This process is described in detail in U.S. Pat. No. 5,885,211. That device facilitates a rapid and painless method of eliminating the barrier function of the stratum corneum to facilitate the transcutaneous transport of therapeutic substances into the body when applied topically or to access the analytes within the body for analysis. The method utilizes a procedure that begins with the contact application of a small area heat source to the targeted area of the stratum corneum or other selected biological membrane.

In particular microprojection arrays such as those described in U.S. Pat. Nos. 6,855,372; 7,097,631; and 7,131,960 and Published U.S. Patent Application Nos. US2005/10031676; US2005/0049549; US2006/0030811 and US2007/0299388 may be used for transdermal delivery of compositions comprising a gap junction modulation agent, e.g., an anti-connexin compound, for pain relief.

In one embodiment, the piercing elements of microprojection arrays have a projection length less than 1000 microns. In a further embodiment, the piercing elements have a projection length of less than 500 microns, more preferably, less than 250 microns. The microprojections further have a width in the range of approximately 25-500 microns and a thickness in the range of approximately 10-100 microns. The microprojections may be formed in different shapes, such as, for example, needles, blades, pins, punches, and combinations thereof.

Formulations suitable for coating microprojection arrays for transdermal delivery after therapeutic agents are described in U.S. Pat. No. 6,855,372 and Published Patent Application Nos. US2005/0256045; US2007/0184096 and US2008/0039775. The gap junction modulation compounds, such as the anti-connexin compounds described herein, may be formulated as described therein and used to coat microprojection arrays for the transdermal delivery of the anti-connexin compounds.

In some aspects, the coating formulations have a viscosity less than approximately 500 centipoise and greater than about 3 centipoise.

In one embodiment, the thickness of the biocompatible coating is less than about 25 microns, more preferably, less than about 10 microns, as measured from the microprojection surface.

The desired coating thickness may be dependent upon several factors, including the required dosage of gap junction modulation agent and, hence, coating thickness necessary to deliver the dosage, the density of the microprojections per unit area of the sheet, the viscosity and concentration of the coating composition and the coating method chosen.

In accordance with one embodiment, the method for delivering a gap junction modulation agent contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from about 5 seconds up to about 24 hours. Following the desired wearing time, the microprojection member is removed.

Preferably, the amount of gap junction modulation agent contained in the biocompatible coating (i.e., dose) is in the range of approximately 1 μg-1000 more preferably, in the range of approximately 10-200 μg per dosage unit. Even more preferably, the amount of gap junction modulation agent contained in the biocompatible coating is in the range of approximately 10-100 μg per dosage unit. Higher doses are also contemplated, for example, up to 2, 3, 4, 5, 6, 7, 8, 9 and 10 milligrams or more, as are repeat applications of doses as needed or desired for pain relief.

After a coating has been applied, the coating formulation is dried onto the microprojections by various means. In a non-limiting preferred embodiment, the coated microprojection member is dried in ambient room conditions. However, various temperatures and humidity levels can be used to dry the coating formulation onto the microprojections. Additionally, the coated member can be heated, lyophilized, freeze dried or similar techniques used to remove the water from the coating.

### Compositions for Transdermal Delivery

The described embodiments can be organized according to their ability to deliver a low or high molecular weight gap junction modulation agent. Low molecular weight molecules (e.g., a molecule having a molecular weight less than 6,000 daltons) can be effectively delivered using an embodiment of the invention and high molecular weight molecules (e.g., a molecule having a molecular weight greater than 6,000 daltons) can be effectively delivered using an embodiment of the invention. In one embodiment, a transdermal delivery system described herein provides a therapeutically or effective amount of a delivered agent having a molecular weight of 50 daltons to less than 6,000 daltons. Preferably, however, a transdermal delivery system described herein provides a therapeutically or effective amount of a gap junction modulation agent having a molecular weight of 50 daltons to 2,000,000 daltons or less.

In some embodiments, a gap junction modulation agent is transdermally delivered to cells in the body using an embodiment of the transdermal delivery system described herein.

### Penetration Enhancers

A penetration enhancer included in many embodiments of the invention comprises two components—a hydrophobic component and a hydrophilic component. Desirably, the hydrophobic component comprises a polyether compound, such as an ethoxylated vegetable, nut, synthetic, or animal oil, which has the ability to reduce the surface tension of materials that are dissolved into it. Not wanting to be tied to any particular mechanism or mode of action and offered only to expand the knowledge in the field, it is contemplated that the attachment of poly (ethylene oxide) to the components of a particular oil occurs not on a particular functional group but rather the polyethylene oxide chains begin to grow from unsaturated C.dbd.C bonds and from the occasional glycerol unit. Because an ethoxylated oil, such as ethoxylated macadamia nut oil, is a mixture of various fatty acids, fatty alcohols, and fatty amines, the components of the oil may have varying amounts of ethoxylation. Accordingly, measurements of ethoxylation/molecule (e.g., 16 ethoxylations/molecule) are an average of the amount of ethoxylation present on the components of the oil rather than on any specific component itself.

Non-limiting preferred ethoxylated oils can be obtained or created from, for example, macadamia nut oil, meadowfoam, castor oil, jojoba oil, corn oil, sunflower oil, sesame oil, and emu oil. Many of these oils are commercially available from Floratech of Gilbert, Ariz. or other suppliers. Alternatively, ethoxylated oils can be prepared by reacting the oil with ethylene oxide. Pure carrier oils that are suitable for ethoxylation so as to create a penetration enhancer for use with the transdermal delivery systems described herein are described in U.S. Pat. Nos. 7,220,427; 7,300,666; and 7,316,820, the disclosures of which are incorporated herein by reference, and can be obtained from Esoteric oils Pty. Ltd., Pretoria South Africa.

In some embodiments a reduction in the number of ethoxylations on a light oil may produce a superior transdermal delivery product. This was unexpected because as the amount of ethoxylations on a molecule of oil decreases its miscibility with the aqueous components of the delivery system decreases.

Other compounds often found in ethoxylated oils that may be beneficial for some embodiments and methods described herein are glycerol-polyethylene glycol ricinoleate, the fatty esters of polyethylene glycol, polyethylene glycol, and ethoxylated glycerol. Some of these compounds exhibit hydrophilic properties and the hydrophilic-lipophilic balance (HLB) is preferably maintained between 10 and 18. Any number of methods have been devised to characterize HLB, but perhaps the most widely used is the octanol/water coefficient. (See Calculating log Poct from Structures”, by Albert J. Leo, Chemical Reviews, vol 93, pp 1281).

Accordingly, some of the components of the oils in the table above and related fatty acids, fatty alcohols, and fatty amines can be ethoxylated and used as a penetration enhancer or to enhance another penetration enhancer (e.g., ethoxylated macadamia nut oil). For example, some embodiments comprise a penetration enhancer that consists of, consists essentially of, or comprises ethoxylated palmitoleic acid, ethoxylated oleic acid, ethoxylated gondoic acid, or ethoxylated erucic acid. These compounds can be prepared synthetically or isolated or purified from oils that contain large quantities of these fatty acids and the synthesized, isolated, or purified fatty acids can then be reacted with ethylene oxide.

Recent research reports have indicated that Aloe Vera, a term used to describe the extract obtained from processing the entire leaf, isolated from the Aloe Vera species of Aloe, can be used as a vehicle for delivering hydrocortisone, estradiol, and testosterone propionate. (See Davis, et al, JAPMA 81:1 (1991) and U.S. Pat. No. 5,708,038 to Davis)). As set forth in Davis (U.S. Pat. No. 5,708,308), one embodiment of “Aloe Vera” can be prepared by “whole-leaf processing” of the whole leaf of the Aloe barbadensis plant. Briefly, whole leaves obtained from the Aloe barbadensis plant are ground, filtered, treated with cellulase (optional) and activated carbon and lyophilized. The lyophilized powder is then reconstituted with water prior to use.

### Preparing Transdermal Delivery Systems

In general, transdermal delivery systems are prepared by combining a penetration enhancer with a delivered agent and, optionally, an aqueous adjuvant. Depending on the solubility of the delivered agent, the delivered agent can be solubilized in either the hydrophobic or hydrophilic components of the penetration enhancer. In some formulations, (e.g., formulations containing oil soluble gap junction modulation agents), the delivered agent may readily dissolve in the ethoxylated oil without water, alcohol, or an aqueous adjuvant. In other formulations, the delivered agent may dissolve in water, which is then mixed with the ethoxylated oil. Additionally, some delivered agents can be solubilized in the aqueous adjuvant prior to mixing with the penetration enhancer. Suitably, the pH of the mixture is maintained between 3 and 11 and preferably between 5 and 9.

The transdermal delivery systems described herein can be processed in accordance with conventional pharmacological methods to produce medicinal agents for administration to patients, e.g., mammals including humans. The transdermal delivery systems described herein can be incorporated into a pharmaceutical product with or without modification. The compositions of the invention can be employed in admixture with conventional excipients, e.g., pharmaceutically acceptable organic or inorganic carrier substances suitable for topical application that do not deleteriously react with the molecules that assemble the delivery system. The preparations can be sterilized and if desired mixed with auxiliary agents, e.g., lubricants, preservatives, stabilizers, coloring, aromatic substances and the like that do not deleteriously react with the active compounds. As appropriate, they can also be combined where desired with other active agents.

In some embodiments, the transdermal delivery system is provided as a single dose application containing a pre-measured amount of the delivered agent. For example, septum sealed vials with or without an applicator (e.g., a swab) containing a pre-measured amount of transdermal delivery system (e.g., 0.5 ml) containing a pre-measured amount of a delivered agent are embodiments within the invention. These embodiments have significant utility because pre-determined doses of certain delivered agents facilitate appropriate treatment regimens and the individually sealed doses of the transdermal delivery system with delivered agent maintain sterility of the composition between applications.

### Therapeutic and Prophylactic Applications

Many embodiments are suitable for treatment of subjects either as a preventive measure (e.g., to avoid pain) or as a therapeutic composition to treat subjects who are suffering form acute or chronic pain. In general, many gap junction modulation compounds that can be incorporated into a pharmaceutical formulation can be formulated into a transdermal delivery system of the invention. Because the various formulations of transdermal delivery system described herein have a considerable range in hydrophobic and hydrophilic character, it is suitable for a number gap junction modulation compounds and can be incorporated therein. In addition to transdermal delivery, other forms of administration are suitable. These include, for example, injections, depot injections and instillations, and delivery under the skin and into or in the vicinity of pain, including in a muscle, joint or tendon, or cartilage, as well as intraarticular injections.

In certain embodiments, by adjusting the amount of ethoxylation, alcohol, and water in a particular formulation many agents may be solubilized in a transdermal delivery system. Furthermore, because the transdermal delivery systems described herein can deliver a wide range of gap junction modulation agents, both high and low molecular weight, the transdermal delivery systems described herein have broad utility. The aspects of the invention that follow are for exemplary purposes only, and one of skill in the art can readily appreciate the widespread applicability of a transdermal delivery systems described herein and the incorporation of other delivered agents into a formulation of transdermal delivery system may be done.

In one embodiment, for example, a method of treatment or prevention of pain, including pain associated with an arthritic condition, comprises using a transdermal delivery system described herein that has been formulated with, or includes, a gap junction modulation agent. Arthritic conditions include the various forms of arthritis, including rheumatoid arthritis, osteoarthritis, cervical arthritis and ankylosing spondylitis. Also included is treatment of nerve pain including any pain associated with injury, lesion or dysfunction of a nerve, e.g. neuralgia and neuropathic pain. Nerve pain includes, for example, diabetic nerve pain, sciatic nerve pain, facial nerve pain, nerve injuries, as well as pinched nerves, and fibromyalgia. Exemplary qualities of neuropathic pain may include burning or coldness, “pins and needles” sensations, numbness and itching. Nociceptive pain (e.g. commonly described as aching) is also included. In addition, exemplary nerve pain may also include nerve-pain associated symptoms characterized by, for example, numbness; very sensitive to touch; having an exaggerated pain response; tingling, prickling or burning pain, especially at night; electric, sharp or shooting pain; deep, aching pain; muscle weakness; wasting of muscles. Neuropathic pain may result from disorders of the peripheral nervous system or the central nervous system (brain and spinal cord). Thus, neuropathic pain may be divided into peripheral neuropathic pain, central neuropathic pain, or mixed (peripheral and central) neuropathic pain. Central neuropathic pain may occur in spinal cord injury, multiple sclerosis, and some strokes. Aside from diabetes and other metabolic conditions, neuropathic pain is common in cancer as a direct result of cancer on peripheral nerves (e.g., compression by a tumor), or as a side effect of chemotherapy, radiation injury or surgery. For example, for treatment of body parts such as the arm and/or leg.

By one approach, a transdermal delivery system comprising a gap junction modulation agent that is effective at reducing pain is administered to a subject in need and, optionally, the reduction in pain is monitored. An additional approach involves identifying a subject in need of a gap junction modulation agent (such as an anti-connexin compound) and administering a transdermal delivery system comprising such an agent. The transdermal delivery system is preferably applied to the skin at an area associated with pain or the particular condition and treatment is continued for a sufficient time to reduce pain. Typically, pain may be reduced in 30-60 minutes after application. Relief has generally also been reported within several hours to 1-2 days after application as well. Multiple applications may be given as needed for pain relief. Pain can be acute or chronic, and can be in a supporting body structure or otherwise within the musculoskeletal system.

In one aspect, the invention includes pharmaceutical compositions for transdermal application that are useful for the treatment of a subject for pain, for example, following trauma, as a result of a condition such as an arthritic condition, or prior to, during or following an invasive procedure or surgery, e.g., an orthopedic procedure or surgery, or other condition associated with pain in a supporting body structure or in the musculoskeletal system. Formulations include topical delivery forms and formulations, comprising a pharmaceutically acceptable carrier and therapeutically effective amounts of gap junction modulation agent, for example, an anti-connexin oligonucleotide or peptide or peptidomimetic, alone or in combination with another gap junction modulation agent.

In another aspect, the invention includes pharmaceutical compositions useful for the treatment of a subject for pain comprising a pharmaceutically acceptable carrier and therapeutically effective amounts of a first anti-connexin agent and a second anti-connexin agent as described herein, for example, an anti-connexin polynucleotide and one or more anti-connexin peptides, peptidomimetics, or other gap junction modulation agents. Examples of anti-connexin polynucleotides include anti-connexin oligodeoxynucleotides (“ODN”), including antisense (including modified and unmodified backbone antisense), RNAi, and siRNA. Suitable anti-connexin peptides include connexin binding peptides. Suitable anti-connexin agents include for example, antisense ODNs and other anti-connexin oligonucleotides, peptides and peptidomimetics against connexins 43, 26, and 30, as well as 31.1, 32 and 37. In certain embodiments, suitable compositions include multiple anti-connexin agents in combination, including for example, anti-connexin 43, 26, 30, and 31.1 agents. Non-limiting preferred anti-connexin agents, including anti-connexin oligonucleotides and anti-connexin peptides and peptidomimetics, are directed against connexin 43. Other non-limiting preferred anti-connexin agents, including anti-connexin oligonucleotides and anti-connexin peptides and peptidomimetics, are directed against connexins 26 and 30.

In one embodiment, the present invention provides methods for the treatment of a subject for pain relief during or following (and/or before, as a pretreatment), for example, an orthopedic procedure or surgery or suffering from, predisposed to, or at risk for various orthopedic-related diseases, disorders, or conditions, including arthritic conditions (including rheumatoid arthritis, osteoarthritis, cervical arthritis and ankylosing spondylitis), or other condition associated with pain in a supporting body structure or in the musculoskeletal system or nerve pain, through the use of two or more anti-connexin agents administered simultaneously, separate, or sequentially. In a non-limiting preferred embodiment, the combined use of a first anti-connexin agent and a second anti-connexin agent as described herein, for example, one or more anti-connexin polynucleotides and one or more anti-connexin peptides, peptidomimetics, or gap junction modifying agents has an additive, synergistic or super-additive effect in the treatment of a subject suffering from, predisposed to, or at risk for pain, for example, in a supporting body structure, including pain resulting from various orthopedic-related diseases, disorders, or conditions. In a non-limiting preferred embodiment, the administration of a combined preparation will have fewer administration time points and/or increased time intervals between administrations as a result of such combined use. In another non-limiting preferred embodiment, the combined use of a first anti-connexin agent and a second anti-connexin agent as described herein, for example, one or more anti-connexin polynucleotides and one or more anti-connexin peptides, peptidomimetics, or gap junction modifying agents, allows a reduced frequency of administration. In another non-limiting preferred embodiment, the combined use of a first anti-connexin agent and a second anti-connexin agent as described herein, for example, one or more anti-connexin polynucleotides and one or more anti-connexin peptides, peptidomimetics, or gap junction modifying agents, allows the use of reduced doses of such agents compared to the dose or doses that may be effective when the agent is administered alone. In general, these anti-connexin agent combinations will have improved therapeutic results over administration of single anti-connexin agents.

In another aspect, the invention includes methods for administering a therapeutically effective amount of a first anti-connexin agent and a second anti-connexin agent as described herein, for example, one or more anti-connexin polynucleotides and one or more anti-connexin peptides, peptidomimetics, or gap junction modifying agents, formulated in a delayed release preparation, a slow release preparation, an extended release preparation, a controlled release preparation, and/or in a repeat action preparation to a subject. Such amounts may be administered to treat pain, including pain during or following an orthopedic procedure or surgery or suffering from, predisposed to, or at risk for various orthopedic-related diseases, disorders, or conditions, such as any form of arthritis, including rheumatoid arthritis, osteoarthritis, cervical arthritis and ankylosing spondylitis.

In certain other aspects, the invention also relates to methods of treating a subject for pain relief (for example, during or following—and/or before, as a pretreatment—an orthopedic procedure or surgery, or as resulting from an arthritic condition, including rheumatoid arthritis, osteoarthritis, cervical arthritis and ankylosing spondylitis) comprising administration of (a) a therapeutically effective amount of one or more anti-connexin peptides or peptidomimetics, alone or in combination with one or more gap junction modifying agents and (b) a therapeutically effective amount of one or more anti-connexin polynucleotides. In one embodiment, surgical outcome is improved. In one embodiment, administration is effective to decrease or prevent, in whole or in part, joint contraction in a post-operative subject. In one embodiment, administration is effective to improve recovery time in a post-operative subject. In one embodiment, administration is effective to decrease pain in a post-operative subject. In one embodiment, administration is effective to improve overall recovery result in a post-operative subject. In one embodiment, improved recovery results comprises increased post-operative mobility. In other embodiments, sub-therapeutically effective amounts of one or more anti-connexin polynucleotides and anti-connexin peptides or peptidomimetics, are administered alone or in combination to provide a desired therapeutic effect

In one embodiment, the subject is treated before, during and/or following one of the following surgical procedures: e.g., a release procedure, an arthroscopic procedure, a joint surgery (e.g., hip, shoulder or knee surgery, including replacement procedures). In general, orthopedic surgeries addressed with the inventions described and claimed herein include hand surgery; shoulder and elbow surgery; total joint reconstruction (arthroplasty); foot and ankle surgery; spine surgery; surgical sports medicine; and orthopedic trauma. Thus, for example, orthopedic surgeries include knee arthroscopy and meniscectomy; shoulder arthroscopy and decompression; carpal tunnel release; knee arthroscopy and chondroplasty; removal of support implants; knee arthroscopy and anterior cruciate ligament reconstruction; knee replacement; repair of femoral neck fractures; repair of trochanteric fractures; debridement of skin/muscle/bone/fracture; knee arthroscopy repair of both menisci; hip replacement; shoulder arthroscopy/distal clavicle excision; repair of rotator cuff tendon; repair fracture of radius/ulna; laminectomy; repair of ankle fracture (bimalleolar type); shoulder arthroscopy and débridement; lumbar spinal fusion; repair fracture of the distal radius; low back intervertebral disc surgery; incise finger tendon sheath; repair of ankle fracture (fibula); repair of femoral shaft fracture; repair of trochanteric fracture. Total hip replacement, total shoulder replacement, and total knee replacement are included as well, as is uni-compartment knee replacement, in which only one side of an arthritic knee is replaced, and joint replacements for other joints, including elbow, wrist, ankle, and fingers. Also included in orthopedic surgeries is bone grafting, a surgical procedure that replaces missing bone with material from the patient's own body, or an artificial, synthetic, or natural substitute.

In yet another aspect, the invention provides a method of pain relief when treating a subject, for example, before, during and/or following an orthopedic procedure or surgery or suffering from, predisposed to, or at risk for various orthopedic-related diseases, disorders, or conditions, or other condition associated with pain in a supporting body structure or in the musculoskeletal system, comprising administering to a subject in need thereof a first composition and a second composition, said first composition comprising a therapeutically effective amount of a anti-connexin 43 polynucleotide and said second composition comprising a therapeutically effective amount of an anti-connexin 43 peptide or peptidomimetic. In one embodiment the first composition is administered first. In another embodiment, the second composition is administered first. In a further embodiment, the method further comprises administration of a third composition, wherein the third composition comprises an anti-connexin polynucleotide, peptide, peptidomimetic or gap junction modifying agent. In one embodiment the third composition is administered first.

In one aspect, the invention provides a method for preventing and/or decreasing a joint contracture before, during and/or following an orthopedic procedure or surgery, comprising administering to a subject in need thereof a therapeutically effective amount of a pharmaceutical composition comprising a first anti-connexin agent and a second anti-connexin agent as described herein, for example, one or more anti-connexin polynucleotides and one or more anti-connexin peptides, peptidomimetics, or gap junction modifying agents. In one embodiment, said method comprises administration of two pharmaceutical compositions, the first composition comprising a therapeutically effective amount of one or more anti-connexin polynucleotides and the second pharmaceutical composition comprising a therapeutically effective amount of one or more anti-connexin peptides, peptidomimetics, or gap junction modifying agents. In one embodiment the first composition is administered first. In another embodiment, the second composition is administered first. In a further embodiment, the method further comprises administration of a third composition, wherein the third composition comprises a therapeutically effective amount of an anti-connexin polynucleotide, peptide or peptidomimetic. In one embodiment the third composition is administered first. In one embodiment the third composition is administered first. In one embodiment, the composition is administered to the site of the injury before, at the time of and/or after a release procedure (e.g., forced manipulation, open release, arthroscopic release, or debulking of scar) to prevent the recurrence of abnormal tissue and/or further contracture. In other embodiments, sub-therapeutically effective amounts of anti-connexin agents are used for administration separately or jointly to provide a combined action that is therapeutically effective.

In another aspect, the invention includes an article of manufacture comprising a vessel containing a therapeutically effective amount of an anti-connexin peptide (e.g., a hemichannel blocker), or therapeutically effective amounts of a first anti-connexin agent and a second anti-connexin agent as described herein, for example, one or more pharmaceutically acceptable anti-connexin polynucleotides and one or more pharmaceutically acceptable anti-connexin peptides, peptidomimetics, or gap junction modifying agents and instructions for use, including use for the treatment of a subject as described herein. In other embodiments, sub-therapeutically effective amounts of first and second anti-connexin agents are used to provide a desired therapeutic effect.

The invention includes an article of manufacture comprising packaging material containing one or more dosage forms containing a therapeutically effective amount of an anti-connexin peptide (e.g., a hemichannel blocker), or therapeutically effective amounts of a first anti-connexin agent and a second anti-connexin agent as described herein, for example, one or more anti-connexin polynucleotides and one or more anti-connexin peptides, peptidomimetics, or gap junction modifying agents, wherein the packaging material has a label that indicates that the dosage form can be used for a subject during or following an orthopedic procedure or surgery or suffering from, predisposed to, or at risk for various orthopedic-related diseases, disorders, or conditions, or other condition associated with pain in a supporting body structure or in the musculoskeletal system. In other embodiments, sub-therapeutically effective amounts of first and second anti-connexin agents are used in the preparation of the article of manufacture that together will provide a desired therapeutic effect.

The invention includes a formulation comprising therapeutically effective amounts of a first anti-connexin agent and a second anti-connexin agent as described herein, for example, one or more anti-connexin polynucleotides and one or more anti-connexin peptides, peptidomimetics, or gap junction modifying agents in amounts effective to promote and improve recovery time, improve overall recovery outcome, decrease joint contracture, and/or decrease vascular damage during or following an orthopedic procedure or surgery. Such formulations include, for example, topical delivery forms and formulations, as well as formulations for injection, instillation, and arthroscopic administration.

Non-limiting preferred formulations include, for example, a pharmaceutical composition of the invention which is formulated as a foam, spray or gel. In one embodiment, the gel is a polyoxyethylene-polyoxypropylene copolymer-based gel or a carboxymethylcellulose-based gel. In a non-limiting preferred embodiment, the gel is a pluronic gel. In other embodiments, the invention provide a formulation comprising sub-therapeutically effective amounts of first and second anti-connexin agents that together will provide a desired therapeutic effect.

The invention includes methods for the use of therapeutically effective amounts of compositions comprising a first anti-connexin agent and a second anti-connexin agent as described herein, for example, one or more anti-connexin polynucleotides and one or more anti-connexin peptides, peptidomimetics, or gap junction modifying agents in the manufacture of a medicament for treating a subject prior to, during or following an orthopedic procedure or surgery or suffering from, predisposed to, or at risk for various orthopedic-related diseases, disorders, or conditions, or other conditions associated with pain in a supporting body structure or in the musculoskeletal system. Such medicaments include, for example, topical delivery forms and formulations, as well as formulations for injection, instillation, and arthroscopic administration. Such medicaments include those for the treatment of a subject as disclosed herein. Such medicaments may optionally include reduced therapeutically effective amounts of a first anti-connexin agent and a second anti-connexin agent as described herein compared to amounts administered when such agents are not administered in combination, for example, reduced amounts of one or more anti-connexin polynucleotides and one or more anti-connexin peptides, peptidomimetics, or gap junction modifying agents, as noted herein. In other embodiments, sub-therapeutically effective amounts of anti-connexin agents are used that together will provide a desired therapeutic effect.

The invention includes method of preparing a medicament for treating a subject prior to, during or following an orthopedic procedure or surgery or suffering from, predisposed to, or at risk for various orthopedic-related diseases, disorders, or conditions, or other condition associated with pain in a supporting body structure or in the musculoskeletal system, comprising bringing together and an effective amount of an anti-connexin peptide (e.g., a hemichannel blocker), or a first anti-connexin agent and a second anti-connexin agent as described herein, including, for example, a first composition and a second composition wherein said first composition comprises an effective amount of an anti-connexin polynucleotide and said second composition comprises an effective amount of an anti-connexin peptide or peptidomimetic. Other embodiments preparing medicaments that include first and second compositions comprising therapeutically effective amounts of an anti-connexin polynucleotide, an anti-connexin peptide or peptidomimetic, a gap junction closing compound, a hemichannel closing compound, and/or a connexin carboxy-terminal polypeptide useful for treating a subject during or following an orthopedic procedure or surgery or suffering from, predisposed to or at risk of orthopedic diseases, disorders and/or condition, or other condition associated with pain in a supporting body structure or in the musculoskeletal system. In other embodiments, sub-therapeutically effective amounts of anti-connexin agents to be used in combination are provided that together will provide a desired therapeutic effect.

### Administration of Compositions

The effective dose and method of administration of a carrier system formulation can vary based on the individual patient and the stage of pain or level of pain relief needed, as well as other factors known to those of skill in the art. Although several doses of delivered agents have been indicated above, the therapeutic efficacy and toxicity of such compounds in a delivery system of the invention can be determined by standard pharmaceutical procedures with experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. Data obtained from animal studies may be used in formulating a range of dosages for human use. The dosage of such compounds lies preferably within a range of circulating concentrations that include the ED50 with little or no toxicity. The dosage varies within this range depending upon the dosage form employed, sensitivity of the patient, and the route of administration.

According to certain aspects, the exact dosage is chosen by the individual physician in view of the patient to be treated. Dosages in the range of 1-500 micrograms, and up to 1000 micrograms or more, are suitable, and may be repeated as needed for pain relief. Other higher doses are contemplated, including doses up to 2, 3, 4, 5, 6, 7, 8, 9 and 10 milligrams. Dosage and administration are adjusted to provide sufficient levels of the gap junction modulation agent or to maintain the desired effect. Additional factors that may be taken into account include the severity of the disease state, age, weight and gender of the patient; diet, time and frequency of administration, drug combination(s), reaction sensitivities, and tolerance/response to therapy. Compositions may be administered daily, although less frequent administration is suitable. For example, compositions may be administered every 2, 3 to 4 days, every week, or once every two weeks. Depending on half-life and clearance rate of the particular formulation, and the amount of pain relief provided and its duration, the pharmaceutical compositions of the invention are administered once, twice, three, four, five, six, seven, eight, nine, ten or more times per day, per week, per fortnight, or per month.

Routes of administration of the delivery systems of the invention are primarily topical, although it is desired to administer some embodiments to cells that reside in deep skin layers. Topical administration is accomplished via a topically applied ointment, cream, oil, gel, rinse, etc., containing a delivery system of the invention. Compositions of delivery system-containing compounds suitable for topical application include, but are not limited to, physiologically acceptable ointments, creams, oils, rinses, and gels. In addition to transdermal delivery, other forms of administration are suitable. These include, for example, injections, depot injections and instillations, and delivery under the skin and into or in the vicinity of pain, including in a muscle, joint or tendon, or cartilage, as well as intraarticular injections.

In some embodiments, the mixture of penetration enhancer, aqueous adjuvant, and delivered agent is incorporated into a device that facilitates application. These apparatus generally have a vessel joined to an applicator, wherein a transdermal delivery system of the invention is incorporated in the vessel. Some devices, for example, facilitate delivery by encouraging vaporization of the mixture. These apparatus have a transdermal delivery system of the invention incorporated in a vessel that is joined to an applicator such as a sprayer (e.g., a pump-driven sprayer). These embodiments can also comprise a propellant for driving the incorporated transdermal delivery system out of the vessel. Other apparatus can be designed to allow for a more focused application. A device that facilitates a focused application of a transdermal delivery system of the invention can have a roll-on or swab-like applicator joined to the vessel that houses the transdermal delivery system. Several devices that facilitate the administration of a delivery system of the invention have a wide range of cosmetic or therapeutic applications.

### EXAMPLES

### Example 1

A transdermal delivery formulation was prepared as follows.

An anti-connexin 43 agent solution was first prepared by dissolving an anti-connexin 43 oligonucleotide, namely SEQ ID NO.2 (Agilent, Boulder, Colo.), in PBS (Oxoid, UK. BR0014 Dulbecco “A” tablets) to obtain a 500 micromolar concentration stock solution.

Emu oil was obtained from a commercial farm source in New Zealand.

To prepare a formulation for application to the skin, emu oil was warmed to about 30° C., and 40 microlitres of 500 micromolar stock solution was added to 960 microlitres of oil to prepare a 20 micromolar concentration (200 micrograms per ml) formulation. The mixture was vortexed and then stored at 4° C.

### Example 2

Subject A, a 55 year-old female, scheduled to undergo knee replacement surgery applied by rubbing on 1 ml of the formulation described in Example 1 (containing 200 micrograms of SEQ ID NO.2) onto the opposite knee (scheduled for surgery at a later date) on the night prior to surgery. The knee was wrapped in clingfilm overnight and the subject showered as normal next morning. The subject reported pain relief which continued beyond bed rest period when the knee was being used for mobility whilst supporting the contralateral operated knee. Some “grinding” reappeared in the knee after 7 days but pain relief persisted for about 10 days.

### Example 3

Subject A of Example 2 had a further treatment in which 1 ml of the formulation described in Example 1 was applied to the skin on the lower leg of her surgically operated leg one week after surgery for pain relief. The knee itself was not painful, as it is essentially titanium and ceramic. However, she had pain above and below the surgical site where muscles and tendons had been splayed to allow access for surgery. Following treatment, the subject again reported significant and sustained pain relief in the treated area.

### Example 4

Subject B was a 37-year old female with ankylosing spondylitis that resulted in severe joint pain in the shoulders, knees and lower back. Subject B rubbed 1 ml Emu oil alone onto a painful arthritic knee joint and 1 ml of the formulation described in Example 1 (containing 200 micrograms of SEQ ID NO.2) onto a painful arthritic shoulder joint. The subject reported no relief for the knee, but reported up to 70% or more pain relief in the shoulder, which persisted 7-10 days post treatment.

### Example 5

Several months following treatment Subject B of Example 4 was suffering from extreme arthritic pain in multiple joints due to the cold weather and reported that she was in “agony,” with severe pain in both knees and difficulty walking and climbing stairs, in the lower back (sacral iliac joints, left and right side), and in the left shoulder with limited mobility. The subject applied 1 ml of the formulation described in Example 1 (containing 200 micrograms of SEQ ID NO.2) to all five areas of pain. The formulation was applied prior to retiring to bed and four hours later the subject got out of bed and went for her regular morning walk. Although the subject walked daily to manage her arthritis, she reported that this was uncomfortable and sometimes difficult, especially walking up hills or climbing stairs. Fours hours post-administration of the formulation described in Example 1 to the affected joints she was able to walk freely and reported no pain in either knee, sacral iliac joints, or shoulder. The subject also reported increased mobility in her left shoulder following application of the Example 1 formulation.

Repeat applications of 1 ml of the formulation described in Example 1 (containing 200 micrograms of SEQ ID NO.2) to the shoulder and knee following reoccurrence of pain resulting in complete pain relief for 3-4 days.

### Example 6

Subject C, a 22-year old male, suffered an injury from basketball, believed to be a slight shoulder tear. The subject reported acute pain where the actual tear was believed to be located. The subject reported that this acute pain also caused substantial pain and aching throughout the rest of his shoulder. The subject rubbed 1 ml of the formulation described in Example 1 (containing 200 micrograms of SEQ ID NO.1) was applied to his painful shoulder.

Within an hour of applying the formulation to his shoulder the subject reported that he began to feel the pain relief, and indicated that he achieved about an 80% reduction in acute pain, while the secondary pain being caused by the injury had completely subsided. This pain relief effect remained throughout the day.

On day 2, the subject reported that his shoulder continued to improve. He reported that the acute pain from the injury site became less intense, and confirmed that the secondary pain did not return.

On day 3, the subject reported that he was pain free, and that he had full shoulder joint mobility with no pain.

### Example 7

Subject D, an 81 year-old female with both knees scheduled for replacement, had difficulty walking and was reliant on painkillers. Both knees were treated with 2 ml of the formulation described in Example 1 (containing 400 micrograms of SEQ ID NO.2), each knee wrapped in cling film overnight and washed as normal next morning. On day one post-treatment the subject reported less pain and easier movement. On day 2 the subject reported some pain in right knee (scheduled for replacement) but no pain in left knee. The subject also reported that she had no knee seizures during the night (normally a regular occurrence) and that she slept on her back for the first time in several years. Normally, her knees usually lock up painfully when she sleeps in that position.

The subject also stated that she stopped her Celebrex pain killers (100 mg morning and night) on the morning prior to treatment and has not had to recommence (that is, has not required painkillers for over 48 hours).

### Example 8

Subject E, an 84 year-old male, who was confined to a rest home. The subject had a lower leg (calf) infection thought to have developed from a scratch or similar cause leading to a cellulitis-type condition. The rest home treated occasionally the subject with antibiotics but his condition did not resolve.

0.5 ml of the formulation described in Example 1 (containing 100 micrograms of SEQ ID NO.2), was rubbed on with no obvious effect at 24 hours. Two weeks later the subject's leg flared up again and became swollen, red and sore in an approximately 10-15 cm diameter area and another half ml of the Example 1 formulation was applied to the area. The inflammation and pain had died down completely within 2-3 days and had not returned after 5 five weeks follow-up.

### Example 9

Subject F was a 60 year-old female with chronic knee pain. The subject treated one knee only with 2 ml of the formulation described in Example 1 (containing 200 micrograms of SEQ ID NO.2), and the knee wrapped in cling film overnight and washed as normal next morning. She awoke during the night and reported that she already felt pain relief and that by the next day she was completely pain free, very mobile and able to turn on the knee. When wanting to stand from her desk at work she would usually rise to her feet, pause, and then turn her body (to avoid twisting on the knees) but reported that she was now able to stand, twist and walk freely. She also reported no knee stiffness. The pain relief and flexibility lasted five days before the benefits gradually wore off.

### Example 10

Subject G was a 70 year-old female with a ten year history of joint, muscle and nerve damage sustained during multiple automobile accidents. The subject has undergone numerous treatments for pain in the neck, shoulder, arm, and between spine/shoulder blade, and had been diagnosed as follows: C2-3, no spinal cord involvement, slight left foraminal narrowing with mild facet arthropathy; C3-4 central disk herniation with mild banana shape of the spinal cord but CSF ring present, mild left facet arthropathy with bilateral foraminal narrowing left greater than right; C4-5 central disk herniation with slight ossification, spinal cord draping with narrowing of the subarachnoid space both anteriorly and posteriorly plus bilateral foraminal narrowing with left greater than right facet arthopathy; C5-6 bilateral foraminal narrowing left greater than right facet arthropathy, spinal cord not compressed; C6-7 bilateral foraminal narrowing left greater than right facet arthropathy, spinal cord not compressed; C7-T1 open but s mild right facet arthropathy and questionable osteophyte off the inferior facet joint on the left but the nerve root has adequate space.

The following treatments had been tried at various times over the previous ten years with some but not sustained success: physical therapy, corticoid steroid shots, acupuncture, NSAIDs, and traction.

Treatment with 1 ml of the formulation described in Example 1 (containing 200 micrograms of SEQ ID NO.2) was initiated concomitant with new round of physical therapy. The formulation was mixed by repeated inversion (10×) and applied by gloved hand (finger tip method) to the right shoulder and the back of the neck, and a small remaining portion was used on the left elbow. This treatment was repeated once every two weeks with 4 doses being administered. The subject reported that she has experienced elimination of sharp, stabbing debilitating pain, and that here overall pain has been reduced to where normal daily activities (e.g., cleaning, gardening) have been resumed.

The present invention is not limited by the aforementioned particular preferred embodiments. It will occur to those ordinarily skilled in the art that various modifications may be made to the disclosed preferred embodiments with-out diverting from the concept of the invention. All such modifications are intended to be within the scope of the present invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and applicants reserve the right to revise the application or claims to refer specifically to any individual member or any subgroup of members of the Markush group.

Other embodiments are within the following claims. The patent may not be interpreted to be limited to the specific examples or embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

We claim:

1. A method for reducing pain in a supporting body structure of a subject, comprising topically administering to said subject in need thereof a pharmaceutical composition comprising a therapeutically effective amount of a connexin 43 gap junction modulation agent in a pharmaceutically acceptable transdermal delivery form, whereby pain is reduced.

2. A method according to claim 1, wherein the supporting body structure is a joint.

3. A method according to claim 1, wherein the supporting body structure is selected from the group consisting of muscles, bones, tendons, ligaments and cartilage.

4. A method according to claim 1, wherein the subject is suffering from arthritis.

5. A method according to claim 1 or 2, wherein the subject is suffering from osteoarthritis.

6. A method according to claim 1, wherein the subject is suffering from rheumatoid arthritis.

7. A method according to claim 1, wherein the subject is suffering from cervical arthritis.

8. A method according to claim 1, wherein the subject is suffering from ankylosing spondylitis.

9. A method according to claim 1, wherein the subject is suffering from acute pain.

10. A method according to claim 9, wherein the subject is suffering from back pain, knee pain, hip pain, shoulder pain, hand pain or finger pain.

11. A method according to claim 1, wherein the subject is suffering from chronic pain.

12. A method according to claim 11, wherein the subject is suffering from back pain, knee pain, hip pain, shoulder pain, hand pain or finger pain.

13. A method according to claim 1, wherein the subject is suffering from postoperative pain.

14. A method according to claim 1, wherein the transdermal dosage form is selected from the group consisting of a topical gel, lotion, ointment, or spray.

15. A method according to claim 1, wherein said transdermal delivery form comprises a transdermal penetration agent comprising an oil.

16. A method according to claim 15, wherein the oil is an ethoxylated oil having between 10 and 19 ethoxylations/molecule.

17. A method according to claim 15, wherein said ethoxylated oil contains 16 ethoxylations/molecule.

18. A method according to claim 15, wherein said oil comprises an oil selected from the group consisting of macadamia nut oil, meadowfoam oil, castor oil, jojoba oil, corn oil, sun flower oil, sesame oil and emu oil.

19. A method according to claim 16, wherein said ethoxylated oil comprises an ethoxylated oil selected from the group consisting of macadamia nut oil, meadowfoam oil, castor oil, jojoba oil, corn oil, sun flower oil, sesame oil and emu oil.

20. A method according to claim 15, wherein said oil is an emu oil.

21. A method according to claim 16, wherein said oil ethoxylated is an ethoxylated emu oil.

22. A method according to claim 1, wherein said connexin 43 gap junction modulation agent is 10,000 daltons or greater.

23. A method according to claim 1, wherein said connexin 43 gap junction modulation agent is less than 10,000 daltons.

24. A method according to claim 1, wherein said connexin 43 gap junction modulation agent is an oligonucleotide.

25. A method according to claim 24, wherein said oligonucleotide is selected from the group consisting of an antisense oligonucleotide, a ribozyme, a RNAi oligonucleotide and a siRNA oligonucleotide.

26. A method according to claim 1, wherein said connexin 43 gap junction modulation agent is a connexin 43 antisense oligonucleotide.

27. A method according to claim 26, wherein said antisense oligonucleotide is selected from GTA ATT GCG GCA AGA AGA ATT GTT TCT GTC (SEQ ID NO:1); GTA ATT GCG GCA GGA GGA ATT GTT TCT GTC (SEQ ID NO:2); and, GGC AAG AGA CAC CAA AGA CAC TAC CAG CAT (SEQ ID NO:3).

28. A method according to claim 26, wherein said antisense oligonucleotide has from about 15 to about 35 nucleotides and is sufficiently complementary to connexin 43 mRNA to form a duplex having a melting point greater than 20° C. under physiological conditions.

29. A method according to claim 26, wherein the antisense oligonucleotide has from about 15 to about 35 nucleotides and has at least about 70 percent homology to an antisense sequence of connexin 43 mRNA.

30. A method according to claim 1, wherein said connexin 43 gap junction modulation agent is an RNAi or siRNA polynucleotide.

31. A method according to claim 1, wherein said connexin 43 gap junction modulation agent is a peptide or peptidomimetic.

32. A method according to claim 31, wherein said peptide or peptidomimetic binds to a connexin 43 hemichannel.

33. A method according to claim 31, wherein said peptide or peptidomimetic binds to a connexin 43 ZO-1 protein binding site.

34. A method according to claim 1, further comprising a second pharmaceutical compound, wherein said second pharmaceutical compound is a non-steroidal anti inflammatory drug.

35. A method according to claim 1, wherein said connexin 43 gap junction modulation agent is a connexin 43 phosphorylation agent.

36. A method according to claim 1, wherein the amount of said connexin 43 gap junction modulation agent by weight or volume is from about 0.01% to about 50.0%.

37. A method according to claim 1, wherein said connexin 43 gap junction modulation agent has an approximate average molecular weight of less than about 10,000 daltons and the therapeutically effective amount by weight or volume is about 0.01% to about 50.0%.

38. A method according to claim 1, wherein the therapeutically effective amount of said connexin 43 gap junction modulation agent is about 0.01% to about 10.0%.

39. A method according to claim 1, wherein the therapeutically effective amount of said connexin 43 gap junction modulation agent by weight or volume is about 0.01% to about 5.0%.

40. A method according to claim 1, wherein said composition is administered to an area of skin proximal to a site of tissue or joint pain in the subject.

41. A pharmaceutical composition for reducing pain in a subject, comprising a pain-reducing amount of an anti-connexin 43 compound and a pharmaceutically acceptable vehicle comprising a transdermal delivery agent.

42. A pharmaceutical composition for reducing pain in a supporting body structure of a subject, comprising a formulation having a pain-reducing amount of an anti-connexin 43 compound in a transdermal dosage form.

43. A pharmaceutical composition according to claim 41, wherein said composition comprises a transdermal penetration enhancer.

44. A pharmaceutical composition according to claim 41, wherein said anti-connexin 43 compound is an oligonucleotide and said transdermal penetration agent promotes the delivery of oligonucleotides through the skin.

45. A method for reducing pain in a supporting body structure of a subject, which comprises applying to the subject in need thereof a transdermal delivery device comprising an anti-connexin 43 compound to an area of skin proximal to a site of tissue or joint pain in said subject.

46. A method according to claim 45, wherein the anti-connexin 43 compound is an oligonucleotide and the transdermal delivery device promotes delivery of oligonucleotides through the skin.

47. A method according to claim 46, wherein said transdermal delivery device is a transdermal microprojection delivery device.

48. A method according to claim 47 wherein said microprojection device has a biocompatible coating being formed from a coating formulation having the anti-connexin 43 compound disposed thereon.

49. A method according to claim 46 wherein said transdermal delivery device forms at least one micropore in a tissue membrane whereby delivery of said anti-connexin 43 compound through the skin is promoted.

50. An article of manufacture comprising a packaging material and a transdermal delivery composition contained within said packaging material, wherein said transdermal delivery composition comprises a pain relief effective amount of an anti-connexin 43 compound and a transdermal penetration effective amount of an ethoxylated oil; and wherein said packaging material comprises a label that indicates that said composition may be used for reducing pain in a supporting structure.

51. An article of manufacture according to claim 50 wherein said ethoxylated oil is selected from the group comprising of ethoxylated macadamia nut oil, ethoxylated meadowfoam oil, ethoxylated castor oil, ethoxylated jojoba oil, ethoxylated corn oil, ethoxylated sunflower oil, ethoxylated sesame oil, and ethoxylated emu oil.

52. An article according to claim 50 wherein said anti-connexin 43 compound is an oligonucleotide.

53. An article of manufacture comprising a packaging material and a transdermal delivery composition contained within said packaging material, wherein said transdermal delivery composition comprises a pain relief effective amount of an anti-connexin 43 compound and a transdermal penetration effective amount of an oil; and wherein said packaging material comprises a label that indicates that said composition may be used for reducing pain in a supporting structure.

54. An article of manufacture according to claim 53 wherein said oil is selected from the group comprising of macadamia nut oil, meadowfoam oil, castor oil, jojoba oil, corn oil, sunflower oil, sesame oil, and emu oil.

55. An article according to claim 53 wherein said anti-connexin 43 compound is an oligonucleotide.

56. A method for reducing pain in a supporting body structure or musculoskeletal system of a subject, comprising administering to said subject in need thereof a therapeutically effective amount of a connexin 43 gap junction modulation agent-containing transdermal, injectable, instillation, or depot dosage form, whereby pain is reduced.
